(19) United States
(12) Patent Application Publication
(10) Pub. No.: US 2006/0099692 A1
(43) Pub. Date: May 11, 2006

(54) Microorganisms and processes for enhanced production of pantothenate

(76) Inventors: R Rogers Yocum, Lexington, MA (US); Thomas A Patterson, North Attleboro, MA (US); Janice G Pero, Lexington, MA (US); Theron Hermann, Kinnelon, NJ (US)

Correspondence Address: LAHIVE & COCKFIELD, 28 STATE STREET, BOSTON, MA, 02109, US

(21) Appl. No.: 10/520,210

(22) PCT Filed: Jul. 3, 2002
(86) PCT No.: PCT/US02/21336

§ 371 Date: Sep. 28, 2005

(51) Int. Cl. C12P 19/12; C12P 13/04; C12N 1/21

(52) U.S. Cl. 435/100; 435/106; 435/252.31

(57) ABSTRACT

The present invention features improved methods for the enhanced production of pantoate and pantothenate utilizing microorganisms having modified pantothenate biosynthetic enzyme activities and having modified methylenetetrahydrofolate (MTF) biosynthetic enzyme activities. In particular, the invention features methods for enhancing production of desired products by increasing levels of a key intermediate, ketopantoate, by increasing enzymes or substrates that contribute directly or indirectly to its synthesis. Recombinant microorganisms and conditions for culturing same are also featured. Also featured are compositions produced by such microorganisms.

### RELATED APPLICATIONS

This application is related to International Patent Application No. PCT/US02/00925, entitled “Microorganisms and Processes for Enhanced Production of Pantothenate”, filed Jan. 18, 2002 (pending), and to International Patent Application No. PCT/US00/25993, entitled “Methods and Microorganisms for Production of Panto-Compounds”, filed Sep. 21, 2000 (expired). The entire content of the above-referenced applications is incorporated herein by this reference.

### BACKGROUND OF THE INVENTION

Pantothenate, also known as pantothenic acid or vitamin B5, is a member of the B complex of vitamins and is a nutritional requirement for mammals, including livestock and humans (e.g., from food sources, as a water soluble vitamin supplement or as a feed additive). In cells, panthothenate is used primarily for the biosynthesis of coenzyme A (CoA) and acyl carrier protein (ACP). These coenzymes function in the metabolism of acyl moieties which form thioesters with the sulfhydryl group of the 4′-phosphopantetheine portion of these molecules. These coenzymes are essential in all cells, participating in over 100 different intermediary reactions in cellular metabolism.

The conventional means of synthesizing pantothenate (in particular, the bioactive D isomer) is via chemicals, a process which is hampered by excessive substrate cost as well as the requirement for optical resolution of racemic intermediates. Accordingly, researchers have recently looked to bacterial or microbial systems that produce enzymes useful in pantothenate biosynthesis processes (as bacteria are themselves capable of synthesizing pantothenate). In particular, bioconversion processes have been evaluated as a means of favoring production of the preferred isomer of pantothenic acid. Moreover, methods of direct microbial synthesis have recently been examined as a means of facilitating D-pantothenate production.

There is still, however, significant need for improved pantothenate production processes, in particular, for microbial processes optimized to produce higher yields of desired product

### SUMMARY OF THE INVENTION

The present invention relates to improved processes (e.g., microbial syntheses) for the production of pantothenate. Pantothenate production processes have been described in related applications which feature, for example, microbes engineered to overexpress key enzymes of the pantothenate biosynthetic pathway and the isoleucine-valine biosynthetic pathway (see e.g., FIG. 1). Strains have been. engineered that are capable of producing >50 g/l of pantothenate in standard fermentation processes (see e.g., International Public. No. WO 01/21772 and U.S. Patent Application No. 60/262,995). In particular, increasing the expression of the panB, panC, panD and panE1 genes and increasing the expression of the ilvBNC and ilvD genes results in strains that convert glucose (pyruvate) to commercially attractive quantities of pantothenate.

In order to enhance production levels of for example, pantothenate, various improvements on the above-described methods have now been developed. For example, U.S. patent application Ser. No. 09/667,569 describes production strains having modified (e.g., deleted or decreased-activity) pantothenate kinase enzymes. In such strains, the pantothenate levels are effectively increased by decreasing utilization of pantothenate for coenzymeA (“CoA”) synthesis. U.S. Patent Application Ser. No. 60/262,995 further describes improved pantothenate-production strains that have been engineered to minimize utilization of various pantothenate biosynthetic enzymes and/or isoleucine-valine biosynthetic enzymes and/or their respective substrates from being used to produce an alternative product identified as [R]-3-(2-hydroxy-3-methyl-butyrylamino)-propionic acid (“HMBPA”).

The present invention features methods to further enhance pantothenate production by modulating a biosynthetic pathway that supplies a substrate for the pantothenate biosynthetic pathway, namely the methylenetetrahydrofolate (“MTF”) biosynthetic pathway. In particular, it has been discovered that increasing levels of MTF by modification of the MTF biosynthetic pathway results in enhanced levels of the key pantothenate biosynthetic pathway intermediate, ketopantoate. Enhanced ketopantoate levels, in turn, result in significantly enhanced pantothenate production levels in appropriately engineered strains. In essence, the present inventors have identified a limiting step in the productioon of panto-compounds (e.g., pantothenate) by strains engineered to overexpress, for example, the panB, panC, panD, panE1, ilvBNC and ilvD genes, and describe herein a means for overcoming this limitation by modification of the MTF biosynthetic pathway.

At least three effective means of modifying the MTF biosynthetic pathway are described herein. In one aspect, it has been demonstrated that increasing serine levels in the culture medium of pantothenate-producing microorganisms results in enhanced panto-compound production. It has also been demonstrated that increasing the synthesis or activity of 3-phosphoglycerate dehydrogenase (the serA gene product) or the synthesis or activity of serine hydroxymethyl transferase (the glyA gene product), thereby enhancing serine and methylenetetrahydrofolate biosynthesis in appropriately engineered microorganisms, increases panto-compound production. Increased synthesis of 3-phosphoglycerate dehydrogenase (the serA gene product) is achieved, for example, by overexpressing serA from an appropriately-engineered expression cassette. Increased synthesis of serine hydroxymethyl transferase (the glyA gene product) is achieved, for example, by overexpressing glyA from an appropriately-engineered expression cassette. Alternatively, levels of serine hydroxymethyl transferase (the glyA gene product) are increased by altering the regulation of the glyA gene. For example, mutation or deletion of the gene encoding a negative regulator (i.e., repressor) of glyA expression, the purR gene, effectively increases glyA expression. Additional methods suitable for increasing MTF levels in panto-compound producing microorganisms involve deregulating enzymes responsible for converting glycine to MTF (e.g., glycine cleavage enzymes).

Accordingly, in one aspect the invention features processes for the enhanced production of pantoate and pantothenate that involve culturing microorganisms having modified pantothenate biosynthetic enzyme activities and having modified methylenetetrahydrofolate (MTF) biosynthetic enzyme activities under conditions such that pantothenate production is enhanced. In another aspect the invention features processes for the enhanced production of pantoate and pantothenate that involve culturing microorganisms having modified pantothenate biosynthetic enzyme activities, having modified isoleucine-valine (ilv) biosynthetic enzymes, and having modified methylenetetrahydrofolate (MTF) biosynthetic enzyme activities under conditions such that pantothenate production is enhanced. In particular, the invention features methods for enhancing production of desired products (e.g., pantoate and/or pantothenate) by increasing the levels of a key intermediate, ketopantoate, by enzymes that contribute to its synthesis. Preferred methods result in production of pantothenate at levels greater than 50, 60, 70 or more g/L after 36 hours of culturing the microorganisms, or such that at least 60, 70, 80, 90, 100, 110, 120 or more g/L pantothenate is produced after 36 hours of culturing the microorganisms. Recombinant microorganisms and conditions for culturing same are also are featured. Also featured are compositions produced by such microorganisms.

Other features and advantages of the invention will be apparent from the following detailed description and claims.

### BRIEF DESCRIPTION OF THE DRAWINGS

FIG. 1 is a schematic representation of the pantothenate and isoluecine-valine (ilv) biosynthetic pathways. Pantothenate biosynthetic enzymes are depicted in bold and their corresponding genes indicated in italics. Isoleucine-valine (ilv) biosynthetic enzymes are depicted in bold italics and their corresponding genes indicated in italics.

FIG. 2 is a schematic representation of the methylenetetrahydrofolate (“MTF”) biosynthetic pathway in E. coli (and presumably in B. subtilis).

FIG. 3 is a schematic representation of the construction of the plasmid pAN665.

FIG. 4 is a schematic representation of the construction of the plasmid pAN670.

FIG. 5 is a schematic representation of the plasmid pAN004.

FIG. 6 is a schematic representation of the plasmid pAN396.

FIG. 7 is a schematic representation of the plasmid pAN393.

FIG. 8 is a schematic representation of the structure of pAN835F, a clone of the B. subtilis purR gene.

FIG. 9 is a schematic representation of the structure of pAN838F, a plasmid designed to install a disruption of the B. subtilis purR gene.

FIG. 10 is a schematic representation of the structure of pAN821, a plasmid designed to delete a portion of the serA gene, selecting for kanamycin resistance.

FIG. 11 is a schematic representation of the structure of pAN824, a plasmid designed to integrate a non-amplifiable P₂₆ serA cassette at the serA locus, selecting for Ser⁺.

FIG. 12 is a schematic representation of the structure of pAN395, a medium copy plasmid designed to integrate and amplify a P26 serA expression cassette at the serA locus.

### DETAILED DESCRIPTION OF THE INVENTION

The present invention is directed to improved methods for producing panto-compounds (e.g., ketopantoate, pantoate and/or pantothenate) and stains engineered for use in said improved methods. Strains capable of producing >50 g/l of pantothenate can be constructed as taught in International Patent Application Ser. No. WO 01/21772 and in U.S. Patent Application Ser. No. 60/262,995. By increasing the expression of the panB, panC, panD and panE1 genes and by increasing the expression of the ilvBNC and ilvD genes, one can design strains (e.g., Bacillus strains) that convert glucose (pyruvate) to commercially attractive quantities of pantothenate.

However, it has now been discovered that in strains engineered to express high levels of the panB gene product, ketopantoate hydroxymethyltransferase (e.g., PA824, described in U.S. patent application Ser. No. 09/667,569 and PA668-24, described in U.S. Patent Application Ser. No. 60/262,995), a limiting step for further increases in the production of pantothenate is still the conversion of α-ketoisovalerate (α-KIV) to ketopantoate. Methods to increase the synthesis of α-KIV were described previously in International Patent Application Ser. No. WO 01/21772 and U.S. Patent Application Ser. No. 60/262,995. Here we disclose that even further increases in pantothenate production can be achieved by engineering panto-compound producing microorganisms such that the level of MTF, or the rate of MTF synthesis is enhanced or increased.

Accordingly, the present invention features methods for improving panto-compound production that involve modulating the methylenetetrahydrofolate (“MTF”). biosynthetic pathway. In particular, increasing MTF levels in panto-compound producing microbes is an effective means of enhancing ketopantoate production, and in turn results in enhanced pantoate and/or pantothenate production in appropriately-engineered recombinant microorganisms.

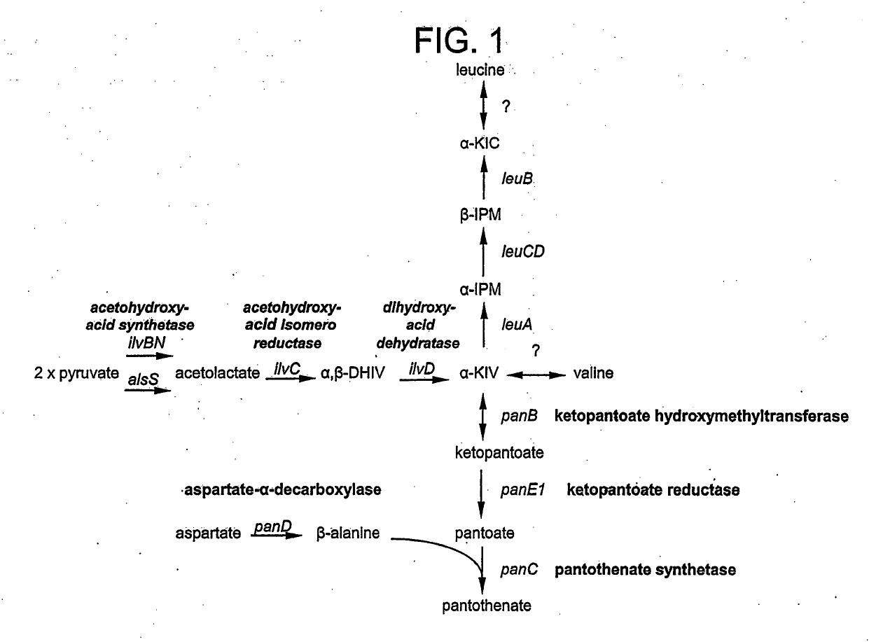

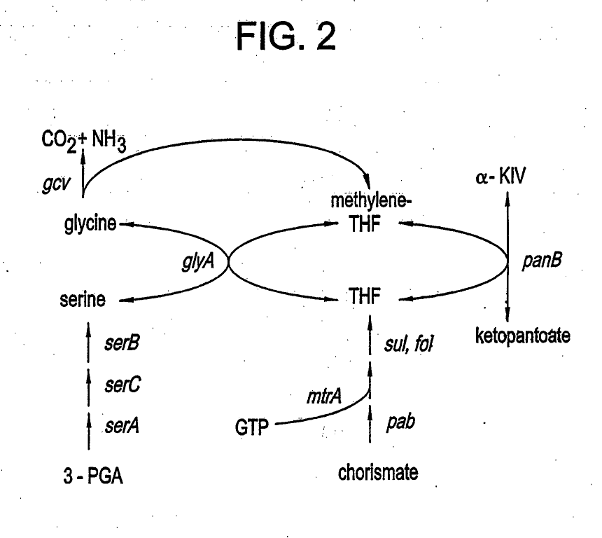

Ketopantoate hydroxymethylenetransferase catalyzes the production of ketopantoate from α-ketoisovalerate (“α-KIV”) and MTF (see e.g., FIG. 1). In particular, the enzyme catalyzes the transfer of a hydroxymethyl group from MTF to α-KIV to yield ketopantoate. Both α-KIV and MTF are substrates for this reaction, and their syntheses can be increased in order to improve production of ketopantoate. The pathway for MTF biosynthesis in E. coli (and also in Bacillus subtilis) is outlined in FIG. 2. MTF is synthesized from tetrahydrofolate and serine in a reaction catalyzed by the glyA gene that encodes serine hydroxymethyl transferase. For improved MTF synthesis the cells need increased quantities of both substrates and the product of the glyA gene.

In one embodiment, the invention features processes for the enhanced production of pantothenate that involve culturing a microorganism having (i) a deregulated pantothenate biosynthetic pathway (e.g., having one, two, three or four pantothenate biosynthetic enzymes deregulated) and (ii) a deregulated methylenetethrhydrofolate (MTF) biosynthetic pathway (e.g., having at least one or two MTF biosynthetic enzymes deregulated), under conditions such that pantothenate production is enhanced. Exemplary pantothenate biosynthetic enzymes include ketopantoate hydroxymethyltransferase, ketopantoate reductase, pantothenate synthetase and aspartate-α-decarboxylase. Exemplary MTF biosynthetic enzymes include the serA gene product and the glyA gene product.

In another embodiment, the invention features processes for the enhanced production of pantothenate that involve culturing a microorganism having (i) a deregulated pantothenate biosynthetic pathway (e.g. having one, two, three or four pantothenate biosynthetic enzymes deregulated), (ii) a deregulated isoleucine-valine (ilv) biosynthetic pathway (e.g., having one, two or three ilv biosynthetic enzymes deregulated), and (iii) a deregulated MTF biosynthetic pathway (e.g., having at least one or two MTF biosynthetic enzymes deregulated), under conditions such that pantothenate production is enhanced. Exemplary ilv biosynthetic enzymes include acetohydroxyacid acid synthetase, acetohydroxyacid isomeroreductase, and dihydroxyacid dehydratase.

In another embodiment, the invention features processes for the production of pantothenate that involve culturing a microorganism having a deregulated pantothenate biosynthetic pathway, a deregulated ilv biosynthetic pathway, and a deregulated MTF biosynthetic pathway, such that at least 50 g/L pantothenate is produced after 36 hours of culturing the microorganism, preferably such that at least 60 g/L pantothenate is produced after 36 hours of culturing the microorganism, more preferably such that at least 70 g/L pantothenate is produced after 36 hours of culturing the microorganism, and most preferably such that at least 80 g/L pantothenate, at least 90 g/L pantothenate, at least 100 g/L pantothenate, at least 110 g/L pantothenate, or at least 120 g/L pantothenate (or more) is produced after 36 hours of culturing the microorganism.

In another embodiment, the invention features processes for the production of pantothenate that involve culturing a microorganism having a deregulated pantothenate biosynthetic pathway, a deregulated ilv biosynthetic pathway, and a deregulated MTF biosynthetic pathway, deregulated such that at least 70 g/L pantothenate is produced after 48 hours of culturing the microorganism, preferably such :that at least 80 g/L pantothenate is produced after 48 hours of culturing the microorganism, and more preferably such that at least 90 g/L pantothenate is produced after 48 hours of culturing the microorganism.

In one exemplary embodiment, deregulation of the MTF biosynthetic pathway is achieved by deregulating the serA gene product in a panto-compound producing strain, for example, by expressing the serA gene constitutively or by introducing a feedback resistant allele of serA. In another exemplary embodiment, deregulation of the MTF biosynthetic pathway is achieved by deregulating the glyA gene product in a panto-compound producing strain, for example, by overexpressing the glyA gene or modulating repression of the glyA gene by mutating or disrupting the purR gene product. In other exemplary embodiments, MTF biosynthesis is modulated by increasing serine in the culture medium or deregualting glycine cleavage enzymes.

The invention further features methods as described above, wherein pantothenate production is further enhanced by regulating pantothenate kinase activity (e.g., wherein pantothenate kinase activity is decreased). In one embodiment, CoaA is deleted and CoaX is downregulated. In another embodiment, CoaX is deleted and CoaA is downregulated. In yet another embodiment, CoaX and CoaA are downregulated. The invention further features methods as described above, wherein the microorganisms are cultured under conditions of excess serine. The invention further features methods as described above, wherein the microorganisms have the pantothenate biosynthetic pathway deregulated such that pantothenate production is independent of β-alanine feed.

Products synthesized according to the processes of the invention are also featured, as are compositions that include pantothenate produced according to said processes. Recombinant microorganisms for use in the processes of the invention are also featured. In one embodiment, the invention features a recombinant microorganism for the enhanced production of pantothenate having a deregulated pantothenate biosynthetic pathway and a deregulated MTF biosynthetic pathway. In another embodiment, the invention features a recombinant microorganism for the enhanced production of pantothenate having a deregulated pantothenate biosynthetic pathway, a deregulated MTF biosynthetic pathway and a deregulated ilv pathway. Microorganisms can further have reduced pantothenate kinase activity. Preferred microorganisms belong to the genus Bacillus, for example Bacillus subtilis.

As described above, certain aspects of the invention feature processes for the enhanced production of panto-compounds (e.g., pantoate and/or pantothenate) that involve culturing microorganisms having at least a deregulated pantothenate biosynthetic pathway. The term “pantothenate biosynthetic pathway” includes the biosynthetic pathway involving pantothenate biosynthetic enzymes (e.g., polypeptides encoded by biosynthetic enzyme-encoding genes), compounds (e.g., substrates, intermediates or products), cofactors and the like utilized in the formation or synthesis of pantothenate. The term “pantothenate biosynthetic pathway” includes the biosynthetic pathway leading to the synthesis of pantothenate in microorganisms (e.g., in vivo) as well as the biosynthetic pathway leading to the synthesis of pantothenate in vitro.

As used herein, a microorganism “having a deregulated pantothenate biosynthetic pathway” includes a microorganism having at least one pantothenate biosynthetic enzyme deregulated (e.g., overexpressed) (both terms as defined herein) such that pantothenate production is enhanced (e.g., as compared to pantothenate production in said microorganism prior to deregulation of said biosynthetic enzyme or as compared to a wild-type microorganism). The term “pantothenate” includes the free acid form of pantothenate, also referred to as “pantothenic acid” as well as any salt thereof (e.g., derived by replacing the acidic hydrogen of pantothenate or pantothenic acid with a cation, for example, calcium, sodium, potassium, ammonium, magnesium), also referred to as a “pantothenate salt”. The term “pantothenate” also includes alcohol derivatives of pantothenate. Preferred pantothenate salts are calcium pantothenate or sodium pantothenate. A preferred alcohol derivative is pantothenol. Pantothenate salts and/or alcohols of the present invention include salts and/or alcohols prepared via conventional methods from the free acids described herein. In another embodiment, a pantothenate salt is synthesized directly by a microorganism of the present invention. A pantothenate salt of the present invention can likewise be converted to a free acid form of pantothenate or pantothenic acid by conventional methodology. The term “pantothenate” is also abbreviated as “pan” herein.

Preferably, a microorganism “having a deregulated pantothenate biosynthetic pathway” includes a microorganism having at least one pantothenate biosynthetic enzyme deregulated (e.g., overexpressed) such that pantothenate production is 1 g/L or greater. More preferably, a microorganism “having a deregulated pantothenate biosynthetic pathway” includes a microorganism having at least one pantothenate biosynthetic enzyme deregulated (e.g., overexpressed) such that pantothenate production is 2 g/L or greater. Even more preferably, a microorganism “having a deregulated pantothenate biosynthetic pathway” includes a microorganism having at least one pantothenate biosynthetic enzyme deregulated (e.g., overexpressed) such that pantothenate production is 10 g/L, 20 g/L, 30 g/L, 40 g/L, 50 g/L, 60 g/L, 70 g/L, 80 g/L, 90 g/L, or greater.

The term “pantothenate biosynthetic enzyme” includes any enzyme utilized in the formation of a compound (e.g., intermediate or product) of the pantothenate biosynthetic pathway. For example, synthesis of pantoate from α-ketoisovalerate (α-KIV) proceeds via the intermediate, ketopantoate. Formation of ketopantoate is catalyzed by the pantothenate biosynthetic enzyme PanB or ketopantoate hydroxymethyltransferase (the panB gene product). Formation of pantoate is catalyzed by the pantothenate biosynthetic enzyme PanE1 or ketopantoate reductase (the panE1 gene product). Synthesis of β-alanine from aspartate is catalyzed by the pantothenate biosynthetic enzyme PanD or aspartate-α-decarboxylase (the panD gene product). Formation of pantothenate from pantoate and β-alanine (e.g., condensation) is catalyzed by the pantothenate biosynthetic enzyme PanC or pantothenate synthetase (the panC gene product). Pantothenate biosynthetic enzymes may also perform an alternative function as enzymes in the HMBPA biosynthetic pathway described herein.

Accordingly, in one embodient, the invention features a process for the enhanced production of pantothenate that includes culturing a microorganism having at least one pantothenate biosynthetic enzyme deregulated (e.g. deregulated such that pantothenate production is enhanced), said enzyme being selected, for example, from the group consisting of PanB (or ketopantoate hydroxymethyltransferase), PanC (or pantothenate synthetase), PanD (or aspartate-α-decarboxylase), PanE1 (or ketopantoate reductase). In another embodiment, the invention features a process for the enhanced production of pantothenate that includes culturing a microorganism having at least two pantothenate biosynthetic enzymes deregulated, said enzymes being selected, for example, from the group consisting of PanB (or ketopantoate hydroxymethyltransferase), PanC (or pantothenate synthetase), PanD (or aspartate-α-decarboxylase), and PanE1 (or ketopantoate reductase). In another embodiment, the invention features a process for the enhacned production of pantothenate that includes culturing a microorganism having at least three pantothenate biosynthetic enzymes deregulated, said enzymes being selected, for example, from the group consisting of PanB (or ketopantoate hydroxymethyltransferase), PanC (or pantothenate synthetase), PanD (or aspartate-α-decarboxylase), and PanE1 (or ketopantoate reductase). In another embodiment, the invention features a process for the enhanced production of pantothenate that includes culturing a microorganism having at least four pantothenate biosynthetic enzymes deregulated, for example, a microorganism having PanB (or ketopantoate hydroxymethyltransferase), PanC (or pantothenate synthetase), PanD (or aspartate-α-decarboxylase), and PanE1 (or ketopantoate reductase) deregulated.

In another aspect, the invention features processes for the enhanced production of pantothenate that involve culturing microorganisms having a deregulated isoleucine-valine biosynthetic pathway. The term “isoleucine-valine biosynthetic pathway” includes the biosynthetic pathway involving isoleucine-valine biosynthetic enzymes (e.g., polypeptides encoded by biosynthetic enzyme-encoding genes), compounds (e.g., substrates, intermediates or products), cofactors and the like utilized in the formation or synthesis of conversion of pyruvate to valine or isoleucine. The term “isoleucine-valine biosynthetic pathway” includes the biosynthetic pathway leading to the synthesis of valine or isoleucine in microorganisms (e.g., in vivo) as well as the biosynthetic pathway leading to the synthesis of valine or isoleucine in vitro.

As used herein, a microorganism “having a deregulated isoleucine-valine (ilv) pathway” includes a microorganism having at least one isoleucine-valine (ilv) biosynthetic enzyme deregulated (e.g., overexpressed) (both terms as defined herein) such that isoleucine and/or valine and/or the valine precursor, α-ketoisovaerate (α-KIV) production is enhanced (e.g., as compared to isoleucine and/or valine and/or α-KIV production in said microorganism prior to deregulation of said biosynthetic enzyme or as compared to a wild-type microorganism). FIG. 1 includes a schematic representation of the isoleucine-valine biosynthetic pathway. Isoleucine-valine biosynthetic enzymes are depicted in bold italics and their corresponding genes indicated in italics. The term “isoleucine-valine biosynthetic enzyme” includes any enzyme utilized in the formation of a compound (e.g., intermediate or product) of the isoleucine-valine biosynthetic pathway. According to FIG. 1, synthesis of valine from pyruvate proceeds via the intermediates, acetolactate, α,β-dihydroxyisovalerate (α,β-DHIV) and α-ketoisovalerate (α-KIV). Formation of acetolactate from pyruvate is catalyzed by the isoleucine-valine biosynthetic enzyme acetohydroxyacid synthetase (the ilvBN gene products, or alternatively, the alsS gene product). Formation of α,β-DHIV from acetolactate is catalyzed by the isoleucine-valine biosynthetic enzyme acetohydroxyacid isomeroreductase (the ilvC gene product). Synthesis of α-KIV from α,β-DHIV is catalyzed by the isoleucine-valine biosynthetic enzyme dihydroxyacid dehydratase (the ilvD gene product). Moreover, valine and isoleucine can be interconverted with their respective α-keto compounds by branched chain amino acid transaminases. Isoleucine-valine biosynthetic enzymes may also perform an alternative function as enzymes in the HMBPA biosynthetic pathway described herein.

Accordingly, in one embodient, the invention features a process for the enhanced production of pantothenate that includes culturing a microorganism having at least one isoleucine-valine (ilv) biosynthetic enzyme deregulated (e.g., deregulated such that valine and/or isoleucine and/or α-KIV production is enhanced), said enzyme being selected, for example, from the group consisting of IlvBN, AlsS (or acetohydroxyacid synthetase), IlvC (or acetohydroxyacid isomeroreductase) and IlvD (or dihydroxyacid dehydratase). In another embodiment, the invention features a process for the enhanced production of pantothenate that includes culturing a microorganism having at least two isoleucine-valine (ilv) biosynthetic enzymes deregulated, said enzyme being selected, for example, from the group consisting of IlvBN, AlsS (or acetohydroxyacid synthetase), IlvC (or acetohydroxyacid isomeroreductase) and IlvD (or dihydroxyacid dehydratase). In another embodiment, the invention features a process for the enhanced production of pantothenate that includes culturing a microorganism having at least three isoleucine-valine (ilv) biosynthetic enzymes deregulated, for example, said microorganism having IlvBN or AlsS (or acetohydroxyacid synthetase), IlvC (or acetohydroxyacid isomeroreductase) and IlvD (or dihydroxyacid dehydratase) deregulated.

As mentioned herein, enzymes of the pantothenate biosynthetic pathway and/or the isoleucine-valine (ilv) pathway have been discovered to have an alternative activity in the synthesis of [R]-3-(2-hydroxy-3-methyl-butyrylamino)-propionic acid (“HMBPA”) or the [R]-3-(2-hydroxy-3-methyl-butyrylamino)-propionic acid (“HMBPA”) biosynthetic pathway. The term “[R]-3-2-hydroxy-3-methyl-butyrylamino)-propionic acid (“HMBPA”) biosynthetic pathway” includes the alternative biosynthetic pathway involving biosynthetic enzymes and compounds (e.g., substrates and the like) traditionally associated with the pantothenate biosynthetic pathway and/or isoleucine-valine (ilv) biosynthetic pathway utilized in the formation or synthesis of HMBPA. The term “HMBPA biosynthetic pathway” includes the biosynthetic pathway leading to the synthesis of HMBPA in microorganisms (e.g., in vivo) as well as the biosynthetic pathway leading to the synthesis of HMBPA in vitro.

The term “HMBPA biosynthetic enzyme” includes any enzyme utilized in the formation of a compound (e.g., intermediate or product) of the HMBPA biosynthetic pathway. For example, synthesis of 2-hydroxyisovaleric acid (α-HIV) from α-ketoisovalerate (α-KIV) is catalyzed by the panE1 or panE2 gene product (PanE1 is alternatively referred to herein as ketopantoate reductase) and/or is catalyzed by the ilvC gene product (alternatively referred to herein as acetohydroxyacid isomeroreductase). Formation of HMBPA from β-alanine and α-HIV is catalyzed by the panC gene product (alternatively referred to herein as pantothenate synthetase).

The term “[R]-3-2-hydroxy-3-methyl-butyrylamino)-propionic acid (“HMBPA”)” includes the free acid form of HMBPA, also referred to as “[R]-3-(2-hydroxy-3-methyl-butyrylamino)-propionate” as well as any salt thereof (e.g., derived by replacing the acidic hydrogen of 3-2-hydroxy-3-methyl-butyrylamino)-propionic acid or 3-(2-hydroxy-3-methyl-butyrylamino)-propionate with a cation, for example, calcium, sodium, potassium, ammonium, magnesium), also referred to as a “3-(2-hydroxy-3-methyl-butyrylamino)-propionic acid salt” or “HMBPA salt”. Preferred HMBPA salts are calcium HMBPA or sodium HMBPA. HMBPA salts of the present invention include salts prepared via conventional methods from the free acids described herein. An HMBPA salt of the present invention can likewise be converted to a free acid form of 3-(2-hydroxy-3-methyl-butyrylamino)-propionic acid or 3-(2-hydroxy-3-methyl-butyrylamino)-propionate by conventional methodology.

In preferred embodiments, the invention features processes for the enhanced production of panto-compounds (e.g., pantoate and/or pantothenate) that involve culturing a microorganism having a deregulated methylenetetrahydrofolate (MTF) biosynthetic pathway. The term “methylenetetrahydrofolate (MTF) biosynthetic pathway” refers to the biosynthetic pathway involving MTF biosynthetic enzymes (e.g., polypeptides encoded by biosynthetic enzyme-encoding genes), compounds (e.g., substrates, intermediates or products), cofactors and the like utilized in the formation or synthesis of the PanB substrate, MTF. The term “methylenetetrahydrofolate (MTF) biosynthetic pathway” refers to the biosynthetic pathway leading to the synthesis of MTF in vivo (e.g., the pathway in E. coli, as depicted in FIG. 2) as well as the biosynthetic pathway leading to the synthesis of MTF in vitro. The term “methylenetetrahydrofolate (MTF) biosynthetic enzyme” includes any enzyme utilized in the formation of a compound (e.g., intermediate or product) of the methylenetetrahydrofolate (MTF) biosynthetic pathway.

The present invention is based, at least in part, on the discovery that deregulation of certain MTF biosynthetic enzymes results in enhanced production of MTF. A MTF biosynthetic enzyme, the deregulation of which results in enhanced MTF production, is termed a “MTF biosynthesis-enhancing enzyme”. Exemplary “MTF biosynthesis-enhancing enzymes” are the serA gene product (3-phosphoglycerate dehydrogenase) and the glyA gene product (serine hydroxymethyl transferase). A microorganism “having a deregulated methylenetetrahydrofolate (MTF) biosynthetic pathway”, is a microorganism having at least one MTF biosynthesis-enhancing enzyme deregulated (e.g., overexpressed) such that MTF production or biosynthesis is enhanced (e.g., as compared to MTF production in said microorganism prior to deregulation of said biosynthetic enzyme or as compared to a wild-type microorganism).

In one embodiment, the invention features a process for the enhanced production of panto-compounds (e.g., pantoate and/or pantothenate) that includes culturing a microorganism having a deregulated “methylenetetrahydrofolate (MTF) biosynthetic pathway”, as defined herein. In another embodiment, the invention features a process for the enhanced production of panto-compounds (e.g., pantoate and/or pantothenate) that includes culturing a microorganism having a deregulated MTF biosynthesis-enhancing enzyme. In preferred embodiments, the invention features processes for the enhanced production of panto-compounds (e.g., pantoate and/or pantothenate) that includes culturing a microorganism having a deregulated glyA gene product (serine hydroxymethyl transferase) and/or a deregulated serA gene product (3-phosphoglycerate dehydrogenase).

Yet another aspect of the present invention features processes for the enhanced production of pantothenate that include culturing microorganisms under culture conditions selected to favor pantothenate production, for example, by culturing microorganisms with excess serine (a glyA substrate) in the medium. The term “excess serine” includes serine levels increased or higher that those routinely utilized for culturing the microorganism in question. For example, culturing the Bacillus microorganisms described in the instant Examples is routinely done in the presence of about 0-2.5 g/L serine. Accordingly, excess serine levels can include levels of greater than 2.5 g/L serine, for example, between about 2.5 and 10 g/L serine. Excess serine levels can include levels of greater than 5 g/L serine, for example, between about 5 and 10 g/L serine.

Yet another aspect of the present invention features culturing the microorganisms described herein under conditions such that pantothenate production is further increased, for example, by increasing pantothenate and/or isoleucine-valine (ilv) biosynthetic pathway precursors and/or intermediates as defined herein (e.g. culturing microorganisms in the presence of excess β-alanine, valine and/or α-KIV) or, alternatively, further modifying said microorganisms such that they are capable of producing significant levels of β-alanine in the absence of a β-alanine feed (i.e., β-alanine independent microorganisms, as described in U.S. patent application Ser. No. 09/09/667,569).

Yet another aspect of the invention features further regulating pantothenate kinase activity in pantothenate-producing strains such that pantothenate production is enhanced. Pantothenate kinase is a key enzyme catalyzing the formation of Coenzyme A (CoA) from pantothenate (see e.g., U.S. patent application Ser. No. 09/09/667,569). Regulation of pantothenate kinase (e.g., decreasing the activity or level of pantothenate kinase) reduces the production of CoA, favoring pantothenate accumulation. In one embodiment, pantotheante kinase activity is decreased by deleting CoaA and downregulating CoaX activity (CoaA and CoaX are both capable of catalyzing the first step in CoA biosynthesis in certain microorganisms). In another embodiment, pantothenate kinase activity is decreased by deleting CoaX and downregulating CoaA. In yet another embodiment, pantotheante kinase activity is decreased by downregulating CoaA and CoaX activities.

Various aspects of the invention are described in further detail in the following subsections.

I. Targeting Genes Encoding Various Pantothenate and/or Isoleucine-Valine (ilv) and/or Methylenetetrahydrofolate (MTF) Biosynthetic Enzymes

In one embodiment, the present invention features modifying or increasing the level of various biosynthetic enzymes of the pantothenate and/or isoleucine-valine (ilv) and/or methylenetetrahydrofolate (MTF) biosynthetic pathways. In particular, the invention features modifying various enzymatic activities associated with said pathways by modifying or altering the genes encoding said biosynthetic enzymes.

The term “gene”, as used herein, includes a nucleic acid molecule (e.g., a DNA molecule or segment thereof) that, in an organism, can be separated from another gene or other genes, by intergenic DNA (i.e., intervening or spacer DNA which naturally flanks the gene and/or separates genes in the chromosomal DNA of the organism). Alternatively, a gene may slightly overlap another gene (e.g., the 3′ end of a first gene overlapping the 5′ end of a second gene), the overlapping genes separated from other genes by intergenic DNA. A gene may direct synthesis of an enzyme or other protein molecule (e.g., may comprise coding seqeunces, for example, a contiguous open reading frame (ORF) which encodes a protein) or may itself be functional in the organism. A gene in an organism, may be clustered in an operon, as defined herein, said operon being separated from other genes and/or operons by the intergenic DNA. An “isolated gene”, as used herein, includes a gene which is essentially free of sequences which naturally flank the gene in the chromosomal DNA of the organism from which the gene is derived (i.e., is free of adjacent coding sequences that encode a second or distinct protein, adjacent structural sequences or the like) and optionally includes 5′ and 3′ regulatory sequences, for example promoter sequences and/or terminator sequences. In one embodiment, an isolated gene includes predominantly coding sequences for a protein (e.g., sequences which encode Bacillus proteins). In another embodiment, an isolated gene includes coding sequences for a protein (e.g., for a Bacillus protein) and adjacent 5′ and/or 3′ regulatory sequences from the chromosomal DNA of the organism from which the gene is derived (e.g., adjacent 5′ and/or 3′ Bacillus regulatory sequences). Preferably, an isolated gene contains less than about 10 kb, 5 kb, 2 kb, 1 kb, 0.5 kb, 0.2 kb, 0.1 kb, 50 bp, 25 bp or 10 bp of nucleotide sequences which naturally flank the gene in the chromosomal DNA of the organism from which the gene is derived.

The term “operon” includes at least two adjacent genes or ORFs, optionally overlapping in sequence at either the 5′ or 3′ end of at least one gene or ORF. The term “operon” includes a coordinated unit of gene expression that contains a promoter and possibly a regulatory element associated with one or more adjacent genes or ORFs (e.g, structural genes encoding enzymes, for example, biosynthetic enzymes). Expression of the genes (e.g., structural genes) can be coordinately regulated, for example, by regulatory proteins binding to the regulatory element or by anti-termination of transcription. The genes of an operon (e.g., structural genes) can be transcribed to give a single mRNA that encodes all of the proteins.

A “gene having a mutation” or “mutant gene” as used herein, includes a gene having a nucleotide sequence which includes at least one alteration (e.g., substitution, insertion, deletion) such that the polypeptide or protein encoded by said mutant exhibits an activity that differs from the polypeptide or protein encoded by the wild-type nucleic acid molecule or gene. In one embodiment, a gene having a mutation or mutant gene encodes a polypeptide or protein having an increased activity as compared to the polypeptide or protein encoded by the wild-type gene, for example, when assayed under similar conditions (e.g., assayed in microorganisms cultured at the same temperature). As used herein, an “increased activity” or “increased enzymatic activity” is one that is at least 5% greater than that of the polypeptide or protein encoded by the wild-type nucleic acid molecule or gene, preferably at least 5-10% greater, more preferably at least 10-25% greater and even more preferably at least 25-50%, 50-75% or 75-100% greater than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, an “increased activity” or “increased enzymatic activity” can also include an activity that is at least 1.25-fold greater than the activity of the polypeptide or protein encoded by the wild-type gene, preferably at least 1.5-fold greater, more preferably at least 2-fold greater and even more preferably at least 3-fold, 4-fold, 5-fold, 10-fold, 20-fold, 50-fold, 100-fold greater than the activity of the polypeptide or protein encoded by the wild-type gene.

In another embodiment, a gene having a mutation or mutant gene encodes a polypeptide or protein having a reduced activity as compared to the polypeptide or protein encoded by the wild-type gene, for example, when assayed under similar conditions (e.g., assayed in microorganisms cultured at the same temperature). A mutant gene also can encode no polypeptide or have a reduced level of production of the wild-type polypeptide. As used herein, a “reduced activity” or “reduced enzymatic activity” is one that is at least 5% less than that of the polypeptide or protein encoded by the wild-type nucleic acid molecule or gene, preferably at least 5-10% less, more preferably at least 10-25% less and even more preferably at least 25-50%, 50-75% or 75-100% less than that of the polypeptide or protein encoded by the wild-type nucleic acid molecule or gene. Ranges intermediate to the above-recited values, e.g., 75-85%, 85-90%, 90-95%, are also intended to be encompassed by the present invention. As used herein, a “reduced activity” or “reduced enzymatic activity” can also include an activity that has been deleted or “knocked out” (e.g., approximately 100% less activity than that of the polypeptide or protein encoded by the wild-type nucleic acid molecule or gene).

Activity can be determined according to any well accepted assay for measuring activity of a particular protein of interest. Activity can be measured or assayed directly, for example, measuring an activity of a protein in a crude cell extract or isolated or purified from a cell or microorganism. Alternatively, an activity can be measured or assayed within a cell or microorganism or in an extracellular medium. For example, assaying for a mutant gene (i.e., said mutant encoding a reduced enzymatic activity) can be accomplished by expressing the mutated gene in a microorganism, for example, a mutant microorganism in which the enzyme is a temperature-sensitive, and assaying the mutant gene for the ability to complement a temperature sensitive (Ts) mutant for enzymatic activity. A mutant gene that encodes an “increased enzymatic activity” can be one that complements the Ts mutant more effectively than, for example, a corresponding wild-type gene. A mutant gene that encodes a “reduced enzymatic activity” is one that complements the Ts mutant less effectively than, for example, a corresponding wild-type gene.

It will be appreciated by the skilled artisan that even a single substitution in a nucleic acid or gene sequence (e.g., a base substitution that encodes an amino acid change in the corresponding amino acid sequence) can dramatically affect the activity of an encoded polypeptide or protein as compared to the corresponding wild-type polypeptide or protein. A mutant gene (e.g., encoding a mutant polypeptide or protein), as defined herein, is readily distinguishable from a nucleic acid or gene encoding a protein homologue in that a mutant gene encodes a protein or polypeptide having an altered activity, optionally observable as a different or distinct phenotype in a microorganism expressing said mutant gene or producing said mutant protein or polypeptide (i.e., a mutant microorganism) as compared to a corresponding microorganism expressing the wild-type gene. By contrast, a protein homologue can have an identical or substantially similar activity, optionally phenotypically indiscernable when produced in a microorganism, as compared to a corresponding microorganism expressing the wild-type gene. Accordingly it is not, for example, the degree of sequence identity between nucleic acid molecules, genes, protein or polypeptides that serves to distinguish between homologues and mutants, rather it is the activity of the encoded protein or polypeptide that distinguishes between homologues and mutants: homologues having, for example, low (e.g., 30-50% sequence identity) sequence identity yet having substantially equivalent functional activities, and mutants, for example sharing 99% sequence identity yet having dramatically different or altered functional activities.

It will also be appreciated by the skilled artisan that nucleic acid molecules, genes, protein or polypeptides for use in the instant invention can be derived from any microorganisms having a MTF biosynthetic pathway, an ilv biosynthetic pathway or a pantothenate biosynthetic pathway. Such nucleic acid molecules, genes, protein or polypeptides can be identified by the skilled artisan using known techniques such as homology screening, sequence comparison and the like, and can be modified by the skilled artisan in such a way that expression or production of these nucleic acid molecules, genes, protein or polypeptides occurs in a recombinant microorganism (e.g., by using appropriate promotors, ribosomal binding sites, expression or integration vectors, modifying the sequence of the genes such that the transcription is increased (taking into account the preferable codon usage), etc., according to techniques described herein and those known in the art).

In one embodiment, the genes of the present invention are derived from a Gram positive microorganism organism (e.g., a microorganism which retains basic dye, for example, crystal violet, due to the presence of a Gram-positive wall surrounding the microorganism). The term “derived from” (e.g., “derived from” a Gram positive microorganism) refers to a gene which is naturally found in the microorganism (e.g., is naturally found in a Gram positive microorganism). In a preferred embodiment, the genes of the present invention are derived from a microorganism belonging to a genus selected from the group consisting of Bacillus, Cornyebacterium (e.g, Cornyebacterium glutamicum), Lactobacillus, Lactococci and Streptomyces. In a more preferred embodiment, the genes of the present invention are derived from a microorganism is of the genus Bacillus. In another preferred embodiment, the genes of the present invention are derived from a microorganism selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus halodurans, and other Group 1 Bacillus species, for example, as characterized by 16S rRNA type. In another preferred embodiment, the gene is derived from Bacillus brevis or Bacillus stearothermophilus. In another preferred embodiment, the genes of the present invention are derived from a microorganism selected from the group consisting of Bacillus licheniformis, Bacillus amyloliquefaciens, Bacillus subtilis, and Bacillus pumilus. In a particularly preferred embodiment, the gene is derived from Bacillus subtilis (e.g., is Bacillus subtilis-derived). The term “derived from Bacillus subtilis” or “Bacillus subtilis-derived” includes a gene which is naturally found in the microorganism Bacillus subtilis. Included within the scope of the present invention are Bacillus-derived genes (e.g., B. subtilis-derived genes), for example, Bacillus or B. subtilis purR genes, serA genes, glyA genes, coaX genes, coaA genes, pan genes and/or ilv genes.

In another embodiment, the genes of the present invention are derived from a Gram negative (excludes basic dye) microorganism. In a preferred embodiment, the genes of the present invention are derived from a microorganism belonging to a genus selected from the group consisting of Salmonella (e.g., Salmonella typhimurium), Escherichia, Klebsiella, Serratia, and Proteus. In a more preferred embodiment, the genes of the present invention are derived from a microorganism of the genus Escherichia. In an even more preferred embodiment, the genes of the present invention are derived from Escherichia coli. In another embodiment, the genes of the present invention are derived from Saccharomyces (e.g., Saccharomyces cerevisiae).

II. Recombinant Nucleic Acid Molecules and Vectors

The present invention further features recombinant nucleic acid molecules (e.g., recombinant DNA molecules) that include genes described herein (e.g., isolated genes), preferably Bacillus genes, more preferably Bacillus subtilis genes, even more preferably Bacillus subtilis pantothenate biosynthetic genes and/or isoleucine-valine (ilv) biosynthetic genes and/or methylenetetrahydrofolate (MTF) biosynthetic genes. The term “recombinant nucleic acid molecule” includes a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). Preferably, a recombinant nucleic acid molecule (e.g., a recombinant DNA molecule) includes an isolated gene of the present invention operably linked to regulatory sequences. The phrase “operably linked to regulatory sequence(s)” means that the nucleotide sequence of the gene of interest is linked to the regulatory sequence(s) in a manner which allows for expression (e.g., enhanced, increased, constitutive, basal, attenuated, decreased or repressed expression) of the gene, preferably expression of a gene product encoded by the gene (e.g., when the recombinant nucleic acid molecule is included in a recombinant vector, as defined herein, and is introduced into a microorganism).

The term “regulatory sequence” includes nucleic acid sequences which affect (e.g., modulate or regulate) expression of other nucleic acid sequences (i.e., genes). In one embodiment, a regulatory sequence is included in a recombinant nucleic acid molecule in a similar or identical position and/or orientation relative to a particular gene of interest as is observed for the regulatory sequence and gene of interest as it appears in nature, e.g., in a native position and/or orientation. For example, a gene of interest can be included in a recombinant nucleic acid molecule operably linked to a regulatory sequence which accompanies or is adjacent to the gene of interest in the natural organism (e.g., operably linked to “native” regulatory sequences (e.g. to the “native” promoter). Alternatively, a gene of interest can be included in a recombinant nucleic acid molecule operably linked to a regulatory sequence which accompanies or is adjacent to another (e.g., a different) gene in the natural organism. Alternatively, a gene of interest can be included in a recombinant nucleic acid molecule operably linked to a regulatory sequence from another organism. For example, regulatory sequences from other microbes (e.g., other bacterial regulatory sequences, bacteriophage regulatory sequences and the like) can be operably linked to a particular gene of interest.

In one embodiment, a regulatory sequence is a non-native or non-naturally-occurring sequence (e.g., a sequence which has been modified, mutated, substituted, derivatized, deleted including sequences which are chemically synthesized). Preferred regulatory sequences include promoters, enhancers, termination signals, anti-termination signals and other expression control elements (e.g., sequences to which repressors or inducers bind and/or binding sites for transcriptional and/or translational regulatory proteins, for example, in the transcribed mRNA). Such regulatory sequences are described, for example, in Sambrook, J., Fritsh, E. F., and Maniatis, T. Molecular Cloning. A Laboratory Manual. 2nd, ed, Cold Spring Harbor Laboratory, Cold Spring Harbor Laboratory Press, Cold Spring Harbor, N.Y., 1989. Regulatory sequences include those which direct constitutive expression of a nucleotide sequence in a microorganism (e.g., constitutive promoters and-strong constitutive promoters), those which direct inducible expression of a nucleotide sequence in a microorganism (e.g., inducible promoters, for example, xylose inducible promoters) and those which attenuate or repress expression of a nucleotide sequence in a microorganism (e.g., attenuation signals or repressor binding sequences, for example, a PurR binding site). It is also within the scope of the present invention to regulate expression of a gene of interest by removing or deleting regulatory sequences. For example, sequences involved in the negative regulation of transcription can be removed such that expression of a gene of interest is enhanced.

In one embodiment, a recombinant nucleic acid molecule of the present invention includes a nucleic acid sequence or gene that encodes at least one bacterial gene product (e.g., a pantothenate biosynthetic enzyme, an isoleucine-valine biosynthetic enzyme and/or a methylenetetrahydrofolate (MTF) biosynthetic enzyme) operably linked to a promoter or promoter sequence. Preferred promoters of the present invention include Bacillus promoters and/or bacteriophage promoters (e.g., bacteriophage which infect Bacillus). In one embodiment, a promoter is a Bacillus promoter, preferably a strong Bacillus promoter (e.g., a promoter associated with a biochemical housekeeping gene in Bacillus or a promoter associated with a glycolytic pathway gene in Bacillus). In another embodiment, a promoter is a bacteriophage promoter. In a preferred embodiment, the promoter is from the bacteriophage SP01. In a particularly preferred embodiment, a promoter is selected from the group consisting of P₁₅, P₂₆ or P_{veg}, having for example, the following respective seqeunces:

| | |
|---|---|
| GCTATTGACGACAGCTATGGTTCACTGTCCACCAAC | (SEQ ID NO:1), |
| CAAAACTGTGCTCAGTACCGCCAATATTTCTCCCTT | |
| GAGGGGTACAAAGAGGTGTCCCTAGAAGAGATCCAC | |
| GCTGTGTAAAAATTTTACAAAAAGGTATTGACTTTC | |
| CCTACAGGGTGTGTAATAATTTAATTACAGGCGGGG | |
| GCAACCCCGCCTGT | |
| GCCTACCTAGCTTCCAAGAAAGATATCCTAACAGCA | (SEQ ID NO:2), |
| CAAGAGCGGAAAGATGTTTTGTTCTACATCCAGAAC | |
| AACCTCTGCTAAAATTCCTGAAAAATTTTGCAAAAA | |
| GTTGTTGACTTTATCTACAAGGTGTGGTATAATAAT | |
| CTTAACAACAGCAGGACGC | |
| and | |
| GAGGAATCATAGAATTTTGTCAAAATAATTTTATTG | (SEQ ID NO:3). |
| ACAACGTCTTATTAACGTTGATATAATTTAAATTTT | |
| ATTTGACAAAAATGGGCTCGTGTTGTACAATAAATG | |
| TAGTGAGGTGGATGCAATG | |

Additional preferred promoters include tef (the translational elongation factor (TEF) promoter) and pyc (the pyruvate carboxylase (PYC) promoter), which promote high level expression in Bacillus (e.g., Bacillus subtilis). Additional preferred promoters, for example, for use in Gram positive microorganisms include, but are not limited to, amy and SPO2 promoters. Additional preferred promoters, for example, for use in Gram negative microorganisms include, but are not limited to, cos, tac, trp, tet, trp-tet, lpp, lac, lpp-lac, lacIQ, T7, T5, T3, gal, trc, ara, SP6, γ-PR or γ-PL.

In another embodiment, a recombinant nucleic acid molecule of the present invention includes a terminator sequence or terminator sequences (e.g., transcription terminator sequences). The term “terminator sequences” includes regulatory sequences that serve to terminate transcription of mRNA. Terminator sequences (or tandem transcription terminators) can further serve to stabilize mRNA (e.g., by adding structure to mRNA), for example, against nucleases.

In yet another embodiment, a recombinant nucleic acid molecule of the present invention includes sequences that allow for detection of the vector containing said sequences (i.e., detectable and/or selectable markers), for example, genes that encode antibiotic resistance sequences or that overcome auxotrophic mutations, for example, trpC, drug markers, fluorescent markers, and/or colorimetric markers (e.g., lacZ/β-galactosidase). In yet another embodiment, a recombinant nucleic acid molecule of the present invention includes an artificial ribosome binding site (RBS) or a sequence that gets transcribed into an artificial RBS. The term “artificial ribosome binding site (RBS)” includes a site within an mRNA molecule (e.g., coded within DNA) to which a ribosome binds (e.g., to initiate translation) which differs from a native RBS (e.g., a RBS found in a naturally-occurring gene) by at least one nucleotide. Preferred artificial RBSs include about 5-6, 7-8, 9-10, 11-12, 13-14, 15-16, 17-18, 19-20, 21-22, 23-24, 25-26, 27-28, 29-30 or more nucleotides of which about 1-2, 3-4, 5-6, 7-8, 9-10, 11-12, 13-15 or more differ from the native RBS (e.g., the native RBS of a gene of interest, for example, the native

| | |
|---|---|
| panB RBS TAAACATGAGGAGGAGAAAACATG | (SEQ ID NO:4) |
| or the native | |
| panD RBS ATTCGAGAAATGGAGAGAATATAA | (SEQ ID NO:5)). |
| TATG | |

Preferably, nucleotides that differ are substituted such that they are identical to one or more nucleotides of an ideal RBS when optimally aligned for comparisons. Ideal RBSs include, but are not limited to,

| | |
|---|---|
| AGAAAGGAGGTGA | (SEQ ID NO:6), |
| TTAAGAAAGGAGGTGANNNNATG | (SEQ ID NO:7), |
| TTAGAAAGGAGGTGANNNNNATG | (SEQ ID NO:8), |
| AGAAAGGAGGTGANNNNNNNATG | (SEQ ID NO:9), |
| and | |
| AGAAAGGAGGTGANNNNNNATG | (SEQ ID NO:10). |

Artificial RBSs can be used to replace the naturally-occurring or native RBSs associated with a particular gene. Artificial RBSs preferably increase translation of a particular gene. Preferred artificial RBSs (e.g., RBSs for increasing the translation of panB, for example, of B. subtilis panB) include

| | |
|---|---|
| CCCTCTAGAAGGAGGAGAAAACATG | (SEQ ID NO:11) |
| and | |
| CCCTCTAGAGGAGGAGAAAACATG | (SEQ ID NO:12). |

Preferred artificial RBSs (e.g., RBSs for increasing the translation of panD, for example, of B. subtilis panD) include

| | |
|---|---|
| TTAGAAAGGAGGATTTAAATATG | (SEQ ID NO:13), |
| TTAGAAAGGAGGTTTAATTAATG | (SEQ ID NO:14), |
| TTAGAAAGGAGGTGATTTAAATG | (SEQ ID NO:15), |
| TTAGAAAGGAGGTGTTTAAAATG | (SEQ ID NO:16), |
| ATTCGAGAAAGGAGGTGAATATAATATG | (SEQ ID NO:17), |
| ATTCGAGAAAGGAGGTGAATAATAATG | (SEQ ID NO:18), |
| and | |
| ATTCGTAGAAAGGAGGTGAATTAATATG | (SEQ ID NO:19). |

The present invention further features vectors (e.g., recombinant vectors) that include nucleic acid molecules (e.g., genes or recombinant nucleic acid molecules comprising said genes) as described herein. The term “recombinant vector” includes a vector (e.g., plasmid, phage, phasmid, virus, cosmid or other purified nucleic acid vector) that has been altered, modified or engineered such that it contains greater, fewer or different nucleic acid sequences than those included in the native or natural nucleic acid molecule from which the recombinant vector was derived. Preferably, the recombinant vector includes a biosynthetic enzyme-encoding gene or recombinant nucleic acid molecule including said gene, operably linked to regulatory sequences, for example, promoter sequences, terminator sequences and/or artificial ribosome binding sites (RBSs), as defined herein. In another embodiment, a recombinant vector of the present invention includes sequences that enhance replication in bacteria (e.g., replication enhancing sequences). In one embodiment, replication-enhancing sequences function in E. coli. In another embodiment, replication-enhancing sequences are derived from pBR322.

In yet another embodiment, a recombinant vector of the present invention includes antibiotic resistance sequences. The term “antibiotic resistance sequences” includes sequences which promote or confer resistance to antibiotics on the host organism (e.g., Bacillus). In one embodiment, the antibiotic resistance sequences are selected from the group consisting of cat (chloramphenicol resistance) sequences, tet (tetracycline resistance) sequences, erm (erythromycin resistance) sequences, neo (neomycin resistance) sequences, kan kanamycin resistence) sequences and spec (spectinomycin resistance) sequences. Recombinant vectors of the present invention can further include homologous recombination sequences (e.g., sequences designed to allow recombination of the gene of interest into the chromosome of the host organism). For example, bpr, vpr, or amyE sequences can be used as homology targets for recombination into the host chromosome. It will further be appreciated by one of skill in the art that the design of a vector can be tailored depending on such factors as the choice of microorganism to be genetically engineered, the level of expression of gene product desired and the like.

III. Recombinant Microorganisms

The present invention further features microorganisms, i.e., recombinant microorganisms, that include vectors or genes (e.g., wild-type and/or mutated genes) as described herein. As used herein, the term “recombinant microorganism” includes a microorganism (e.g., bacteria, yeast cell, fungal cell, etc.) that has been genetically altered, modified or engineered (e.g., genetically engineered) such that it exhibits an altered, modified or different genotype and/or phenotype (e.g., when the genetic modification affects coding nucleic acid sequences of the microorganism) as compared to the naturally-occurring microorganism from which it was derived.

In one embodiment, a recombinant microorganism of the present invention is a Gram positive organism (e.g., a microorganism which retains basic dye, for example, crystal violet, due to the presence of a Gram-positive wall surrounding the microorganism). In a preferred embodiment, the recombinant microorganism is a microorganism belonging to a genus selected from the group consisting of Bacillus, Cornyebacterium (e.g., Cornyebacterium glutamicum), Lactobacillus, Lactococci and Streptomyces. In a more preferred embodiment, the recombinant microorganism is of the genus Bacillus. In another preferred embodiment, the recombinant microorganism is selected from the group consisting of Bacillus subtilis, Bacillus lentimorbus, Bacillus lentus, Bacillus firmus, Bacillus pantothenticus, Bacillus amyloliquefaciens, Bacillus cereus, Bacillus circulans, Bacillus coagulans, Bacillus licheniformis, Bacillus megaterium, Bacillus pumilus, Bacillus thuringiensis, Bacillus halodurans, and other Group 1 Bacillus species, for example, as characterized by 16S rRNA type. In another preferred embodiment, the recombinant microorganism is Bacillus brevis or Bacillus stearothermophilus. In another preferred embodiment, the recombinant microorganism is selected from the group consisting of Bacillus licheniformis, Bacillus amyloliquefaciens, Bacillus subtilis, and Bacillus pumilus.

In another embodiment, the recombinant microorganism is a Gram negative (excludes basic dye) organism. In a preferred embodiment, the recombinant microorganism is a microorganism belonging to a genus selected from the group consisting of Salmonella (e.g., Salmonella typhimurium), Escherichia, Klebsiella, Serratia, and Proteus. In a more preferred embodiment, the recombinant microorganism is of the genus Escherichia. In an even more preferred embodiment, the recombinant microorganism is Escherichia coli. In another embodiment, the recombinant microorganism is Saccharomyces (e.g., Saccharomyces cerevisiae).

A preferred “recombinant” microorganism of the present invention is a microorganism having a deregulated pantothenate biosynthesis pathway or enzyme, a deregulated isoleucine-valine (ilv) biosynthetic pathway or enzyme and/or a modified or deregulated methylenetetrahydrofolate (MTF) biosynthetic pathway or enzyme. The term “deregulated” or “deregulation” includes the alteration or modification of at least one gene in a microorganism that encodes an enzyme in a biosynthetic pathway, such that the level or activity of the biosynthetic enzyme in the microorganism is altered or modified. Preferably, at least one gene that encodes an enzyme in a biosynthetic pathway is altered or modified such that the gene product is enhanced or increased. The phrase “deregulated pathway” includes a biosynthetic pathway in which more than one gene that encodes an enzyme in a biosynthetic pathway is altered or modified such that the level or activity of more than one biosynthetic enzyme is altered or modified. The ability to “deregulate” a pathway (e.g., to simultaneously deregulate more than one gene in a given biosynthetic pathway) in a microorganism in some cases arises from the particular phenomenon of microorganisms in which more than one enzyme (e.g., two or three biosynthetic enzymes) are encoded by genes occurring adjacent to one another on a contiguous piece of genetic material termed an “operon” (defined herein). Due to the coordinated regulation of genes included in an operon, alteration or modification of the single promoter and/or regulatory element can result in alteration or modification of the expression of each gene product encoded by the operon. Alteration or modification of a regulatory element can include, but is not limited to removing the endogenous promoter and/or regulatory element(s), adding strong promoters, inducible promoters or multiple promoters or removing regulatory sequences such that expression of the gene products is modified, modifying the chromosomal location of a gene or operon, altering nucleic acid sequences adjacent to a gene or operon (or within an operon) such as a ribosome binding site, increasing the copy number of a gene or operon, modifying proteins (e.g., regulatory proteins, suppressors, enhancers, transcriptional activators and the like) involved in transcription of a gene or operon and/or translation of a gene product or gene products of a gene or operon, respectively, or any other conventional means of deregulating expression of genes routine in the art (including but not limited to use of antisense nucleic acid molecules, for example, to block expression of repressor proteins). Deregulation can also involve altering the coding region of one or more genes to yield, for example, an enzyme that is feedback resistant or has a higher or lower specific activity.

In another preferred embodiment, a recombinant microorganism is designed or engineered such that at least one pantothenate biosynthetic enzyme, at least one isoleucine-valine biosynthetic enzyme, and/or at least one MTF biosynthetic enzyme is overexpressed. The term “overexpressed” or “overexpression” includes expression of a gene product (e.g., a biosynthetic enzyme) at a level greater than that expressed prior to manipulation of the microorganism or in a comparable microorganism which has not been manipulated. In one embodiment, the microorganism can be genetically designed or engineered to overexpress a level of gene product greater than that expressed in a comparable microorganism which has not been engineered.

Genetic engineering can include, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor proteins). Genetic engineering can also include deletion of a gene, for example, to block a pathway or to remove a repressor.

In another embodiment, the microorganism can be physically or environmentally manipulated to overexpress a level of gene product greater than that expressed prior to manipulation of the microorganism or in a comparable microorganism which has not been manipulated. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

IV. Culturing and Fermenting Recombinant Microorganisms

The term “culturing” includes maintaining and/or growing a living microorganism of the present invention (e.g., maintaining and/or growing a culture or strain). In one embodiment, a microorganism of the invention is cultured in liquid media. In another embodiment, a microorganism of the invention is cultured in solid media or semi-solid media In a preferred embodiment, a microorganism of the invention is cultured in media (e.g., a sterile, liquid medium) comprising nutrients essential or beneficial to the maintenance and/or growth of the microorganism (e.g., carbon sources or carbon substrate, for example carbohydrate, hydrocarbons, oils, fats, fatty acids, organic acids, and alcohols; nitrogen sources, for example, peptone, yeast extracts, meat extracts, malt extracts, soy meal, soy flour, soy grits, urea, ammonium sulfate, ammonium chloride, ammonium nitrate and ammonium phosphate; phosphorus sources, for example, phosphoric acid, sodium and potassium salts thereof; trace elements, for example, magnesium, iron, manganese, calcium, copper, zinc, boron, molybdenum, and/or cobalt salts; as well as growth factors such as amino acids, vitamins, growth promoters and the like).

Preferably, microorganisms of the present invention are cultured under controlled pH. The term “controlled pH” includes any pH which results in production of the desired product (e.g., pantoate and/or pantothenate). In one embodiment microorganisms are cultured at a pH of about 7. In another embodiment, microorganisms are cultured at a pH of between 6.0 and 8.5. The desired pH may be maintained by any number of methods known to those skilled in the art.

Also preferably, microorganisms of the present invention are cultured under controlled aeration. The term “controlled aeration” includes sufficient aeration (e.g., oxygen) to result in production of the desired product (e.g., pantoate and/or pantothenate). In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the cuture vessel (e.g., tube or flask) or by various pumping equipment. Aeration may be further controlled by the passage of sterile air or oxygen through the medium (e.g., through the fermentation mixture). Also preferably, microorganisms of the present invention are cultured without excess foaming (e.g., via addition of antifoaming agents).

Moreover, microorganisms of the present invention can be cultured under controlled temperatures. The term “controlled temperature” includes any temperature which results in production of the desired product (e.g., pantoate and/or pantothenate). In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 50° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In a preferred embodiment, the microorganisms are cultured in shake flasks. In a more preferred embodiment, the microorganisms are cultured in a fermentor (e.g., a fermentation process). Fermentation processes of the present invention include, but are not limited to, batch, fed-batch and continuous processes or methods of fermentation. The phrase “batch process” or “batch fermentation” refers to a system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the exception that one or more substrates or supplements are added (e.g. added in increments or continuously) as the fermentation progresses. The phrase “continuous process” or “continuous fermentation” refers to a system in which a defined fermentation media is added continuously to a fermentor and an equal amount of used or “conditioned” media is simultaneously removed, preferably for recovery of the desired product (e.g. pantoate and/or pantothenate). A variety of such processes have been developed and are well-known in the art.

The phrase “culturing under conditions such that a desired compound is produced” includes maintaining and/or growing microorganisms under conditions (e.g, temperature, pressure, pH, duration, etc.) appropriate or sufficient to obtain production of the desired compound or to obtain desired yields of the particular compound being produced. For example, culturing is continued for a time sufficient to produce the desired amount of a compound (e.g., pantoate and/or pantothenate). Preferably, culturing is continued for a time sufficient to substantially reach suitable production of the compound (e.g. a time sufficient to reach a suitable concentration of pantoate and/or pantothenate or suitable ratio of pantoate and/or pantothenate:HMBPA). In one embodiment, culturing is continued for about 12 to 24 hours. In another embodiment, culturing is continued for about 24 to 36 hours, 36 to 48 hours,48 to 72 hours, 72 to 96 hours, 96 to 120 hours, 120 to 144 hours, or greater than 144 hours. In yet another embodiment, microorganisms are cultured under conditions such that at least about 5 to 10 g/L of compound are produced in about 36 hours, at least about 10 to 20 g/L compound are produced in about 48 hours, or at least about 20 to 30 g/L compound in 20 about 72 hours. In yet another embodiment, microorganisms are cultured under conditions such that at least about 5 to 20 g/L of compound produced in about 36 hours, at least about 20 to 30 g/L compound are produced in about 48 hours, or at least about 30 to 50 or 60 g/L compound in about 72 hours. In yet another embodiment, microorganisms are cultured under conditions such that at least about 40 to 60 g/L of compound are produced in about 36 hours, or at least about 60 to 90. g/L compound are produced in about 48 hours. It will be appreciated by the skilled artisan that values above the upper limits of the ranges recited may be obtainable by the processes described herein, for example, in a particular fermentation run or with a particular engineered strain.

Preferably, a production method of the present invention results in production of a level of pantothenate that is “enhanced as compared to an appropriate control”. The term “appropriate control”, as defined herein, includes any control recognized by the skilled artisan as being approriate for determining enhanced, increased, or elevated levels of desired product. For example, where the process features culturing a microorganism having a deregulated pantothenate biosynthetic pathway and said microorganism further has a deregulated MTF biosynthetic pathway (i.e., has been engineered such that at least one MTF biosynthetic enzyme is deregulated, for example, overexpressed) an appropriate control includes a culture of the microorganism before or absent manipulation of the MTF enzyme or pathway (i.e., having only the pantothenate biosynthetic pathway deregulated). Likewise, where the process features culturing a microorganism having a deregulated pantothenate biosynthetic pathway and a deregulated ilv biosynthetic pathway and said microorganism further has a deregulated MTF biosynthetic pathway (i.e., has been engineered such that at least one MTF biosynthetic enzyme is deregulated, for example, overexpressed) an appropriate control includes a culture of the microorganism before or absent manipulation of the MTF enzyme or pathway (i.e., having only the pantothenate biosynthetic pathway and ilv biosynthetic pathway deregulated). Comparison need not be performed in each process practiced according to the present invention. For example, a skilled artisan can determine appropriate controls empirically from performing a series of reactions (e.g., test tube cultures, shake flask cultures, fermentations), for example, under the same or similar conditions. Having appreciated a routine production level, for example, by a particular strain, the artisan is able to recognize levels that are enhanced, increased or elevated over such levels. In other words, comparison to an appropriate control includes comparison to a predetermined values (e.g., a predetermined control).

Thus, in an embodiment wherein an appropriately engineered strain produces 40 g/L pantothenate in 36 hours (prior to manipulation such that pantothenate production is enhanced), production of ,50, 60, 70 or more g/L pantothenate (after manipulation, for example, manipulation such that at least one MTF biosynthetic enzyme is overexpressed) exemplifies enhanced production. Likewise, in an embodiment wherein an appropriately engineered strain produces 50 g/L pantothenate in 48 hours (prior to manipulation such that pantothenate production is enhanced), production of 60, 70, 80, 90 or more g/L pantothenate (after manipulation, for example, manipulation such that at least one MTF biosynthetic enzyme is overexpressed) exemplifies enhanced production.

The methodology of the present invention can further include a step of recovering a desired compound (e.g., pantoate and or pantothenate). The term “recovering” a desired compound includes extracting, harvesting, isolating or purifying the compound from culture media. Recovering the compound can be performed according to any conventional isolation purification methodology known in the art including, but not limited to, treatment with a conventional resin (e.g., anion or cation exchange resin, non-ionic adsorption resin, etc.), treatment with a conventional adsorbent (e.g. activated charcoal, silicic acid, silica gel, cellulose, alumina, etc.), alteration of pH solvent extraction (e.g., with a conventional solvent such as an alcohol, ethyl acetate, hexane and the like), dialysis, filtration, concentration, crystallization, recrysallization, pH adjustment, lyophilization and the like. For example, a compound can be recovered from culture media by first removing the microorganisms from the culture. Media are then passed through or over a cation exchange resin to remove cations and then through or over an anion exchange resin to remove inorganic anions and organic acids having stronger acidities than the compound of interest. The resulting compound can subsequently be converted to a salt (e.g, a calcium salt) as described herein.

Preferably, a desired compound of the present invention is “extracted”, “isolated” or “purified” such that the resulting preparation is substantially free of other media components (e.g., free of media components and/or fermentation byproducts). The language “substantially free of other media components” includes preparations of the desired compound in which the compound is separated from media components or fermentation byproducts of the culture from which it is produced. In one embodiment, the preparation has greater than about 80% (by dry weight) of the desired compound (e.g., less than about 20% of other media components or fermentation byproducts), more preferably greater than about 90% of the desired compound (e.g., less than about 10% of other media components or fermentation byproducts), still more preferably greater than about 95% of the desired compound (e.g., less than about 5% of other media components or fermentation byproducts), and most preferably greater than about 98-99%. desired compound (e.g., less than about 1-2% other media components or fermentation byproducts). When the desired compound has been derivatized to a salt, the compound is preferably further free of chemical contaminants associated with the formation of the salt. When the desired compound has been derivatized to an alcohol, the compound is preferably further free of chemical contaminants associated with the formation of the alcohol.

In an alternative embodiment, the desired compound is not purified from the microorganism, for example, when the microorganism is biologically non-hazardous (e.g., safe). For example, the entire culture (or culture supernatant) can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized.

In yet another embodiment, the desired compound is partially purified. The term “partially purified” includes media preparations that have had at least some processing, for example, treatment (e.g., batch treatment) with a commercial resin. In preferred embodiments, the “partially purified” preparation has greater than about 30% (by dry weight) of the desired compound, preferably greater than about 40% of the desired compound, more preferably greater than about 50% of the desired compound, still more preferably greater than about 60% of the desired compound, and most preferably greater than about 70% desired compound. “Partially purified” preparations also preferably have 80% or less (by dry weight) of the desired compound (i.e., are less pure than “extracted”, “isolated” or “purified” preparations, as defined herein).

Depending on the biosynthetic enzyme or combination of biosynthetic enzymes manipulated, it may be desirable or necessary to provide (e.g., feed) microorganisms of the present invention at least one biosynthetic precursor such that the desired compound or compounds are produced. The term “biosynthetic precursor” or “precursor” includes an agent or compound which, when provided to, brought into contact with, or included in the culture medium of a microorganism, serves to enhance or increase biosynthesis of the desired product. In one embodiment, the biosynthetic precursor or precursor is aspartate. In another embodiment, the biosynthetic precursor or precursor is β-alanine. The amount of aspartate or β-alanine added is preferably an amount that results in a concentration in the culture medium sufficient to enhance productivity of the microorganism (e.g., a concentration sufficient to enhance production of pantoate and/or pantothenate). Biosynthetic precursors of the present invention can be added in the form of a concentrated solution or suspension (e.g., in a suitable solvent such as water or buffer) or in the form of a solid (e.g., in the form of a powder). Moreover, biosynthetic precursors of the present invention can be added as a single aliquot, continuously or intermittently over a given period of time. The term “excess β-alanine” includes β-alanine levels increased or higher that those routinely utilized for culturing the microorganism in question. For example, culturing the Bacillus microorganisms described in the instant Examples is routinely done in the presence of about 0-0.01 g/L β-alanine. Accordingly, excess β-alanine levels can include levels of about 0.01-1, preferably about 1-20 g/L.

In yet another embodiment, the biosynthetic precursor is valine. In yet another embodiment, the biosynthetic precursor is α-ketoisovalerate. Preferably, valine or α-ketoisovalerate is added in an amount that results in a concentration in the medium sufficient for production of the desired product (e.g., pantoate and/or pantothenate) to occur. The term “excess α-KIV” includes α-KIV levels increased or higher that those routinely utilized for culturing the microorganism in question. For example, culturing the Bacillus microorganisms described in the instant Examples is routinely done in the presence of about 0-0.01 g/L α-KIV. Accordingly, excess α-KIV levels can include levels of about 0.01-1, preferably about 1-20 g/L α-KIV. The term “excess valine” includes valine levels increased or higher that those routinely utilized for culturing the microorganism in question. For example, culturing the Bacillus microorganisms described in the instant Examples is routinely done in the presence of about 0-0.5 g/L valine. Accordingly, excess valine levels can include levels of about 0.5-5 g/L, preferably about 5-20 g/L valine.

In yet another embodiment, the biosynthetic precursor is serine. Preferably, serine is added in an amount that results in a concentration in the medium sufficient for production of the desired product (e.g. pantoate and/or pantothenate) to occur. Excess serine (as defined herein) can also be added according to the production processes described herein, for example, for the enhanced production of pantothenate. The skilled artisan will appreciate that extreme excesses of biosynthetic precursors can result in microorganism toxicity. Biosynthetic precursors are also referred to herein as “supplemental biosynthetic substrates”.

Another aspect of the present invention includes biotransformation processes which feature the recombinant microorganisms described herein. The term “biotransformation process”, also referred to herein as “bioconversion processes”, includes biological processes which results in the production (e.g., transformation or conversion) of appropriate substrates and/or intermediate compounds into a desired product.

The microorganism(s) and/or enzymes used in the biotransformation reactions are in a form allowing them to perform their intended function (e.g. producing a desired compound). The microorganisms can be whole cells, or can be only those portions of the cells necessary to obtain the desired end result. The microorganisms can be suspended (e.g., in an appropriate solution such as buffered solutions or media), rinsed (e.g., rinsed free of media from culturing the microorganism), acetone-dried, immobilized (e.g., with polyacrylamide gel or k-carrageenan or on synthetic supports, for example, beads, matrices and the like), fixed, cross-linked or permeablized (e.g., have permeablized membranes and/or walls such that compounds, for example, substrates, intermediates or products can more easily pass through said membrane or wall).

This invention is further illustrated by the following examples which should not be construed as limiting. The contents of all references, patents and published patent applications cited throughout this application are incorporated herein by reference.

### EXAMPLES

### Example I

### : Panto-Compound Production-Strains

In developing Bacillus strains for the production of pantothenate, various genetic manipulations are made to genes and enzymes involved in the pantothenate biosynthetic pathway and the isoleucine-valine (ilv) pathway (FIG. 1) as described in U.S. patent application Ser. No. 09/400,494 and U.S. patent application Ser. No. 09/667,569. For example, strains having a deregulated panBCD operon and/or having deregulated panE1 exhibit enhanced pantothenate production (when cultured in the presence of β-alanine and α-ketoisovalerate (α-KIV)). Strains further deregulated for ilvBNC and ilvD exhibit enhanced pantothenate production in the presence of only β-alanine. Moreover, it is possible to achieve β-alanine independence by further deregulating panD.

An exemplary pantothenate production strain is PA824, a tryptophan prototroph, Spec and Tet resistant, deregulated for panBCD at the panBCD locus, deregulated for panE1 at the panE1 locus (two genes in the B. subtilis genome are homologous to E. coli panE, panE1 and panE2, the former encoding the major ketopantoate reductase involved in pantothenate production, while panE2 does not contribute to pantothenate synthesis (U.S. patent application Ser. No. 09/400,494), deregulated for ilvD at the ilvD locus, overexpressing an ilvBNC cassette at the amyE locus, and overexpressing panD at the bpr locus. PA824 routinely yields approximately 40-50 g/L pantothenate, when cultured for 48 ours in 14 L fermentor vessels according to standard fermentation procedures (see e.g. provisional patent application Ser. No. 60/263,053 or provisional Patent Application Ser. No. 60/262,995, incorporated by reference herein). Briefly, batch media (4.5 L) containing trace elements is inoculated with shake flask-cultures of PA824. The fermentations are controlled for temperature (e.g., 43° C.), dissolved O₂, and pH, and are run as a- glucose limited fed batch process. After the initial batched glucose is consumed, glucose concentrations are maintained between about 0 and 1 g/L by continuous feeding of fresh FEED media. pH is set at 7.2, monitored, and maintained by feeding either a NH₃- or a H₃PO₄-solution. The dissolved oxygen concentration [pO₂] is maintained at about 10-30% by regulation of the agitation and aeration rate. Foaming is controlled by addition of an appropriate antifoam agent. The pantothenate titer in the fermentation broth is determined (by HPLC analysis) after removal of the cells by centrifugation.

A second exemplary strain is PA668. PA668 is a derivative of PA824 that contains extra copies of P₂₆ panB amplified at the vpr and/or panB locus. PA668 was constructed using a panB expression vector (pAN636) which allows for selection of multiple copies using chloramphenicol. Briefly, a pAN636 NotI restriction fragment (excluding vector sequences) was ligated and then used to transform PA824 with selection on plates containing 5 μg/ml chloramphenicol. Transformants resistant to 30 μg/ml chloramphenicol were isolated and screened for pantothenate production in 48 hour test tube cultures. The isolates produce about 10 percent more pantothenate than PA824. In 10-L fermentations, a first strain, PA668-2A, produces pantothenate in amounts comparable to PA824 cultured under similar conditions (e.g., ˜45-50 g/L at 36 hours). After 36 hours, when pantothenate production routinely begins to slow with PA824, PA668-2A continues to produce significant levels of pantothenate (e.g., ˜60-65 g/l pantothenate at 48 hours). A second strain, PA668-24, produces pantothenate at an even faster rate, reaching 60-70 g/L after 48 hours.

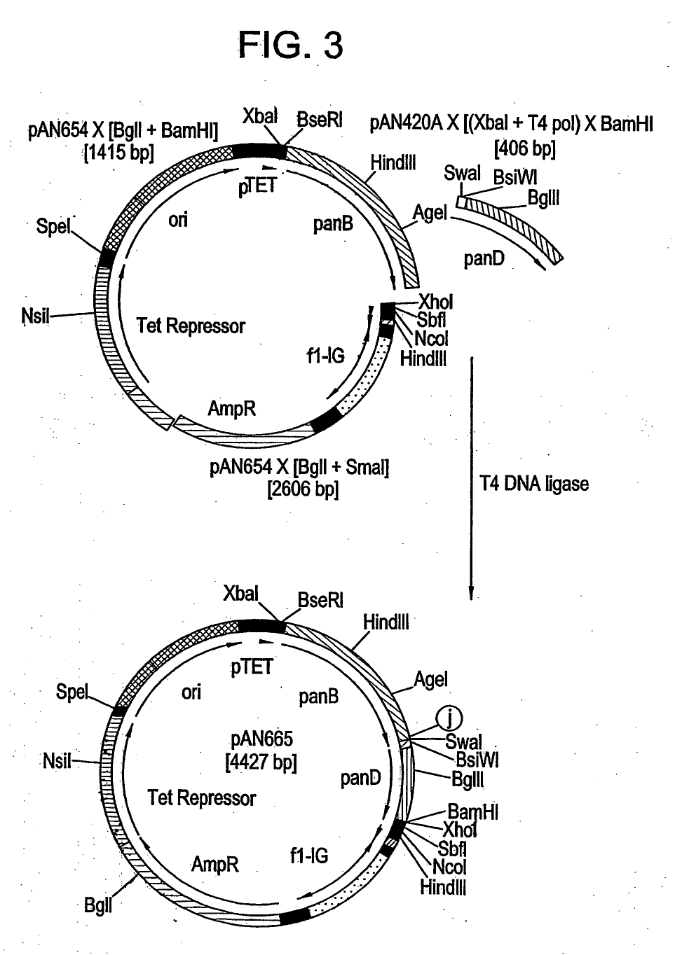

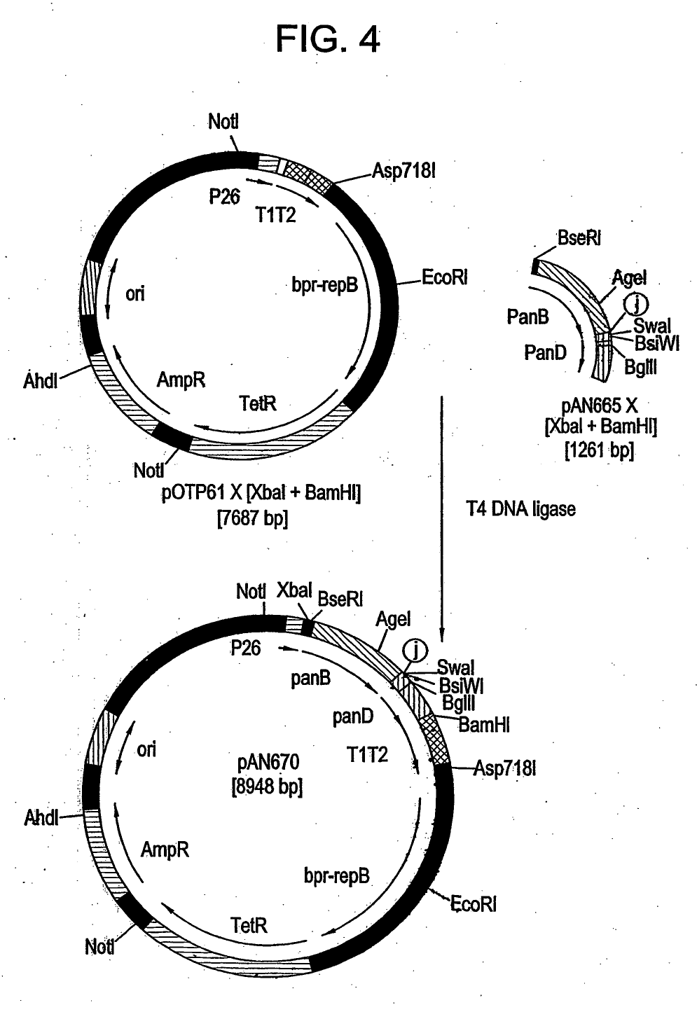

A third production strain, PA721B-39, was engineered to further include an amplifiable P₂₆ panBpanD cassette as follows. First, a single expression cassette was constructed that is capable of integrating both panB and panD at the bpr locus. Combining both genes into one expression cassette simplifies the resulting strain by eliminating an antibiotic resistance marker. The P₂₆ panBpanD expression cassette was constructed to include each of two different panD ribosome binding sites (the RBSs having previously been synthesized and tested in International Public. No. WO 01/21772 and U.S. patent application No. 60/262,995). The further included the synthetic panB gene ribosome binding site (RBS1), but the design permits future alteration of the panB RBS by simple oligonucleotide cassette substitution. In the first step of construction, the panB gene was joined to the two panD gene cassettes as illustrated in FIG. 3 for the construction of pAN665. Next, the resulting panBpanD cassettes were transferred to B. subtilis expression vector pOTP61 as illustrated in FIG. 4. A summary of the essential features of each plasmid (pAN670 and pAN674) constructed is presented in Table 1.

TABLE 1
Plasmids containing various B. subtilis panBpanD gene
expression cassettes.

| Plasmid | panD RBS | Vector | Host strain |
|---|---|---|---|
| pAN665 | Standard | pASK- | E. coli |
| | | 1BA3 | |
| pAN670 | ″ | pOTP61 | B. subtilis |
| pAN669 | ND-C2 | pASK- | E. coli |
| | | 1BA3 | |
| pAN674 | ″ | pOTP61 | B. subtilis |

These new plasmids combine production of extra PanB and PanD from a single vector and were predicted to produce increased levels of PanB relative to the panB expression vector (pAN636) present in PA668. The strategy to install the P26 panBpanD vectors in pantothenate production strains took advantage of genetic linkage between bpr and panE1. A derivative of PA824 was first constructed that is cured of the resident panD expression cassette by transforming the strain with chromosomal DNA isolated from PA930 (panE1::cat) and selecting for resistance to chloramphenicol. The resulting transformants were screened for sensitivity to tetracycline, and two Tet-sensitive isolates named PA715 were saved. This strain is the host strain for testing the P26 panBpanD vectors (see below). In order to restore the P26 panE1 cassette in PA715, each vector was first transformed into a strain (PA328) that contains P26 panE1 but does not contain a cassette integrated at the bpr locus. PA328 does contain the P26 panBCD locus although it is not engineered for overproduction of α-KIV. Transformants of PA328 resistant to tetracycline were obtained using the appropriate NotI restriction fragments from the two vectors and the resulting strains were named PA710 and PA714.

The next step was to transfer the cassettes into PA715 so they could be evaluated in the PA824 strain background. This was accomplished by isolating chromosomal DNA from strains PA710 and PA714 and using each of the two DNAs separately to transform PA715. with selection for resistance to tetracycline. Tetracycline-resistant transformants were screened for sensitivity to chloramphenicol; this identifies the desired transformants that have also acquired the P26 panE1 gene from thee donor DNA by linkage with the P26 panBpanD cassettes at the bpr locus. Chloramphenicol-sensitive isolates derived from transformations in which PA710 or PA714 chromosomal DNA was used as the donor were obtained. The isolates that produced the highest pantothenate titers in test tube culture assays were saved. These strains were named PA717 and PA721, respectively. Duplicate test tube cultures of the new strains, as well as PA824 and PA715, were grown in SVY+10 g/L aspartate at 43° C. for 48 hours and then assayed for pantothenate, HMBPA, and β-alanine. In addition, extracts from each of the strains were run on a SDS-PAGE gel. The results of the test tube culture assays are presented in Table 2.

TABLE 2
Production of pantothenate by strains PA717 and PA721 grown in
SVY plus 10 g/l aspartate.

| | panBD | [pan] | [HMBPA] | [β-ala] |
|---|---|---|---|---|
| Strain | cassette | (g/L) | (g/L) | (g/L) |
| PA824 | — | 4.9 | 0.94 | 2.5 |
| ″ | | 4.6 | 0.79 | 2.3 |
| PA715 | NONE | 1.7 | <0.1 | 0.5 |
| ″ | ″ | 1.7 | <0.1 | 0.4 |
| PA717-24 | pAN670 | 4.8 | 0.34 | 1.3 |
| ″ | ″ | 4.9 | 0.40 | 1.3 |
| PA721-35 | pAN674 | 5.7 | 0.50 | 1.4 |
| ″ | ″ | 5.3 | 0.40 | 1.3 |
| PA721-39 | pAN674 | 4.1 | 0.38 | 2.0 |
| ″ | ″ | 4.6 | 0.40 | 2.2 |

As expected, each of the new strains produced more pantothenate and β-alanine than PA715. Two of the strains (PA717-24 and PA721-39) produced about as much pantothenate as PA824 while PA721-35 produced more pantothenate than PA824. All three of the new strains produced less HMBPA than PA824. The protein gel analysis showed that the three new strains produce more PanB than any of the control strains.

Strains PA717-24, PA721-35, and PA721-39 were also evaluated in shake flask cultures in a soy flour based medium. As shown in Table 3, these strains with the amplifiable P₂₆ panBpanD cassette produced pantothenate and HMBPA at levels similar to the levels seen with PA668-2 and PA668-24 which both contain separate amplifiable P₂₆ panB and P₂₆ panD cassettes.

TABLE 3
Shake Flask Experiment 48 Hours

| | | HMBPA | PAN |
|---|---|---|---|
| Medium | Strain | (g/l) | (g/l) |
| Soy flour + Glucose | PA668-2 | 1.2 | 6.8 |
| | PA668-24 | 1.6 | 5.2 |
| | PA717-24 | 2.0 | 5.9 |
| | PA721-35 | 2.6 | 7.0 |
| | PA721-39 | 2.5 | 8.6 |
| Soy flour + Maltose | PA668-2 | 0.0 | 9.0 |
| | PA668-24 | 0.4 | 10.4 |
| | PA717-24 | 0.7 | 8.6 |
| | PA721-35 | 1.0 | 9.2 |
| | PA721-39 | 0.4 | 9.1 |

Conditions: 40 ml medium/200 ml baffled shake flask, 4× Bioshield covers, 300 rpm, 2.5% inoculum (1.0 ml).
Soy Medium: 20 g/l Cargill 200/20 soy flour, 8 g/l (NH4)2SO4, 5 g/l glutamate, 1× PSTE, 0.1M phosphate pH 7.2 and 0.3M MOPS pH 7.2. 60 g/l glucose or maltose w/10 mM Mg and 1.4 mM Ca.
Average of duplicate flasks.

In addition to producing pantothenate (as well as other panto-compounds depicted in FIG. 1 and described herein), it has been demonstrated that certain strains engineered for producing commercial quantities of desired panto-compound also produce a by-product identified as 3-(2-hydroxy-3-methyl-butyrylamino)-propionic acid (HMBPA) (also referred to as “β-alanine 2-(R)-hydroxyisolvalerate”, “β-alanine 2-hydroxyisolvalerate”, “β-alanyl-α-hydroxyisovalarate” and/or “fantothenate”). (The term “fantothenate” is also abbreviated as “fan” herein.)

HMBPA is the condensation product of [R]-α-hydroxyisovaleric acid (α-HIV) and β-alanine, catalyzed by the PanC enzyme. α-HIV is generated by reduction of α-KIV, a reaction that is catalyzed by the α-keto reductase PanE (e.g., PanE1 and/or PanE2) and/or IlvC. Thus it has been proposed that there exist at least two pathways in microorganisms that compete for α-KIV, the substrate for the biosynthetic enzyme PanB, namely the pantothenate biosynthetic pathway and the HMBPA biosynthetic pathway. (A third and fourth pathway competing for α-KIV are those resulting in the production of valine or leucine from α-KIV, see e.g., FIG. 1). At least the pantothenate biosynthetic pathway and the HMBPA biosynthetic pathway further produce competitive substrates for the enzyme PanC, namely α-HIV and pantoate. Production of HMBPA can have significant effects on pantothenate production. For example, the HMBPA pathway can compete with the pantothenate pathway for precursors (α-KIV and β-alanine) and for some of the enzymes (PanC, PanD, PanE1, and/or IlvC). In addition, because the structure of HMBPA is similar to that of pantothenate, it may have the undesirable property of negatively regulating one or more steps in the pantothenate pathway. Based on the identification of HMBPA, U.S. Provisional Patent Application Ser. No. 60/262,995 teaches that production of pantothenate can be improved or optimized by any means which favor use of substrates (α-KIV and β-alanine) and/or enzymes (PanC, PanD, PanE1, and/or IlvC) in pantothenate biosynthetic processes as compared to HMBPA biosynthetic processes.

### Example II

### Increasing Pantothenate Production by Increasing Serine Availability

At least one method for optimizing pantothenate production involves regulating the availability of serine in the microorganism cultures. In particular, it can be demonstrated that increasing the availability of serine leads to increased pantothenate production (e.g. relative to HMBPA production), whereas decreasing the availability of serine leads to decreased pantothenate production relative to HMBPA production. This method is based on the understanding that the compound, methylenetetrahydrofolate (MTF), which is derived from serine, donates a hydroxymethyl group to α-KIV during the pantothenate biosynthetic reaction to yield ketopantoate (see e.g., FIGS. 1 and 2). Thus, regulating serine levels is one means of effectively regulating ketopantoate levels and, in turn, regulating pantoate and/or pantothenate production in appropriately engineered microorganisms. To demonstrate this regulation, PA824 was grown in test tube cultures of SVY glucose plus 5 g/L β-alanine and ±5 g/L serine for 48 hours and 43° C.

TABLE 4
Production of pantothenate and HMBPA by PA824 with and without
the addition of serine

| serine added | | | |
|---|---|---|---|
| at 5 g/L | OD₆₀₀ | [pan] g/L | [HMBPA] g/L |
| − | 16.3 | 4.9 | 0.84 |
| − | 14.0 | 4.5 | 0.80 |
| + | 13.1 | 6.4 | 0.56 |
| + | 12.9 | 6.0 | 0.62 |

As demonstrated by the data presented in Table 4, addition of serine increases the level of production of pantothenate (while conversely decreasing HMBPA production).

### Example III

### Engineering Bacterial Cells with Increased Amounts of Serine Hydroxylmethyl Transferase, the glyA Gene Product

As an alternative to feeding serine, another method of increasing serine levels and/or serine utilization levels (and accordingly, methylenetetrahydrofolate levels) in order to regulate pantothenate production levels is to increase synthesis or the activity of 3-phosphoglycerate dehydrogenase or of serine hydroxymethyl transferase (the serA and glyA gene products, respectively), thereby increasing serine and methylenetetrahydrofolate biosynthesis in appropriately engineered microorganisms.

Expression of the glyA gene was increased by transforming B. subtilis cells with an expression cassette containing the B. subtilis glyA gene cloned downstream of a strong, constitutive promoter. To construct the expression cassette the primers RY417 and RY418 depicted in Table 5 were used to amplify the glyA gene by PCR from chromosomal DNA isolated from B. subtilis PY79.

TABLE 5

Primers used in the amplification of
B. subtilis glyA and serA

| | | |
|---|---|---|
| RY405 | CCCTCTAGAGGAGGAGAAAACATGTTTCGAG | SEQ ID NO:20 |
| | TATTGGTCTCAGACAAAATG | |
| RY406 | CCCGGATCCAATTATGGCAGATCAATGAGCT | SEQ ID NO:21 |
| | TCACAGACACAA | |
| RY417 | GGATCTAGAGGAGGTGTAAACATGAAACATT | SEQ ID NO:22 |
| | TACCTGCGCAAGACGAA | |
| RY418 | CGGGGATCCCCCATCAACAATTACACACTTC | SEQ ID NO:23 |
| | TATTGATTCTAC | |

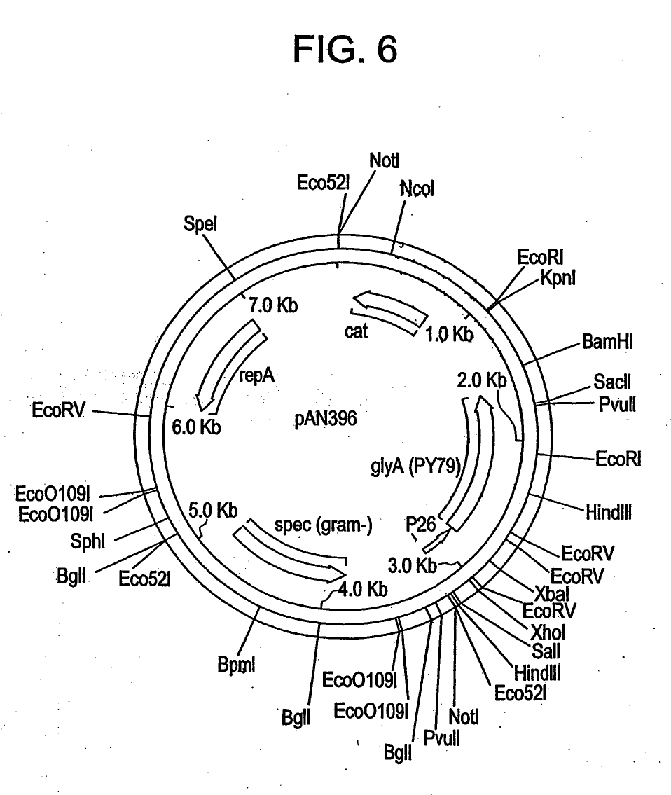

RY417 contains the RBS2 synthetic ribosome binding site just downstream from an XbaI site. The amplified DNA was a then cut with XbaI and BamHI and cloned between the XbaI and BamHI sites in vector pAN004 (FIG. ) to yield plasmid pAN396 (FIG. 6; SEQ ID NO:24). The pAN004 vector contains the phage SP01 P₂₆ promoter immediately upstream of the XbaI cloning site to drive expression of the cloned glyA gene. Just downstream of the expression cassette, pAN396 contains a cat gene that functions in B. subtilis. To transform B. subtilis, the NotI DNA fragment containing the P₂₆ glyA cassette and cat gene was isolated from pAN396, self-ligated, and transformed into competent cells of B. subtilis PY79. Several chloramphenicol resistant transformants were selected and named PA1007 and PA1008. Chromosomal DNA was isolated from each of these strains and used to transform competent cells of PA721B-39 and PA824 to yield strains PA1011 and PA1014, respectively. SDS polyacrylamide gel electrophoresis of cell extracts of selected isolates of PA1011 and PA1014 confirmed that these strains contained increased amounts of the glyA gene product as compared to their parent strains PA721B-39 (described in Example I) and PA824 (described in International Public. No. WO 01/21772). To test the effect of increasing glyA expression on pantothenate production, PA1011 and PA1014 were grown in test tube cultures of SVY glucose plus 5 g/L β-alanine at 43° C. for 48 hours. As shown by the data presented in Table 6, PA1014 produced more pantothenate (4.5 g/L) than its parent strain PA824 (3.2 g/L). Similarly, PA1011 produced on average more pantothenate (4.35 g/L) than its parent strain PA721B-39 (4.05 g/L).

TABLE 6
Production of pantothenate and HMBPA by PA1011 and PA1014
compared to PA721B-39 and PA824.

| | | Pantothenate | HMBPA |
|---|---|---|---|
| Strain | OD₆₀₀ | g/L | g/L |
| PA1014 #1 | 14 | 4.5 | 0.27 |
| PA1014 #2 | 15 | 4.5 | 0.31 |
| PA824 | 16 | 3.1 | 0.31 |
| PA824 | 15 | 3.3 | 0.28 |
| PA1011 #1 | 17 | 4.5 | 0.24 |
| PA1011 #2 | 12 | 4.2 | 0.27 |
| PA721B-39 | 18 | 4.0 | 0.22 |
| PA721B-39 | 16 | 4.1 | 0.25 |

### Example IV

### Engineering Bacterial Cells with Increased Amounts of 3-phosphoglycerate Dehydrogenase, the serA Gene Product

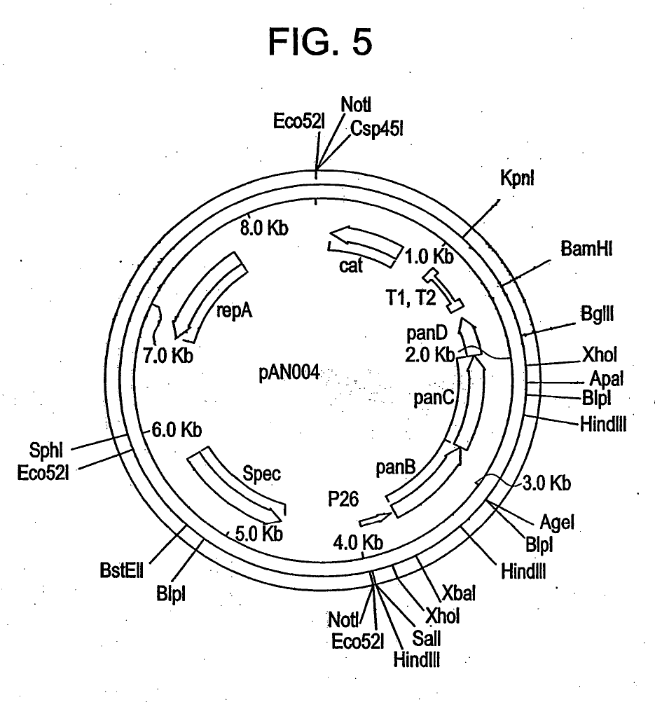

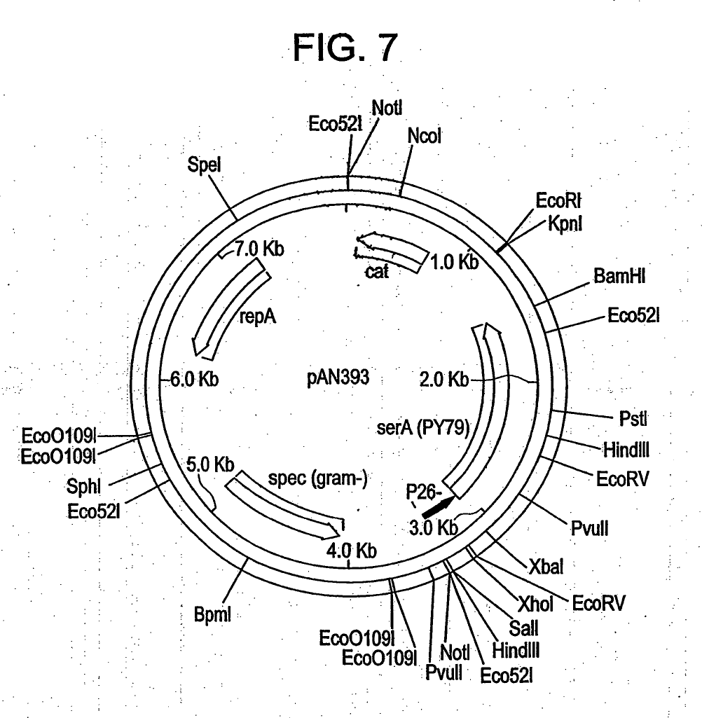

The product of the serA gene, 3-phosphoglycerate dehydrogenase, is the first committed enzyme in the pathway to serine biosynthesis (see FIG. 2). Since serine is one of the substrates for the synthesis of MTF, we engineered the overexpression of the serA gene to increase serine levels in the cell. In a manner similar to that described above for the glyA gene in Example III, expression of the serA gene was increased by transforming B. subtilis cells with an expression cassette containing the B. subtilis serA gene cloned downstream of a strong, constitutive promoter. To construct the expression cassette the primers RY405 and RY406 depicted in Table 5 were used to amplify the serA gene by PCR from chromosomal DNA isolated from B. subtilis PY79. The amplified DNA was then cut with XbaI and BamHI and cloned between the XbaI and BamHI sites in vector pAN004 (FIG. 5) to yield plasmid pAN393 (FIG. 7; SEQ ID NO:25). To transform B. subtilis, the NotI DNA fragment containing the P₂₆ serA cassette and cat gene was isolated from pAN393, self-ligated, and transformed into competent cells of B. subtilis PY79. Several chloramphenicol resistant transformants were selected and named PA1004 and PA1005. Chromosomal DNA was isolated from each of these strains and used to transform competent cells of PA721B-39 and PA824 to yield strains PA1010 and PA1013, respectively. SDS polyacrylamide gel electrophoresis of cell extracts of selected isolates of PA1010 and PA1013 confirmed that these strains contained increased amounts of the serA gene product as compared to their parent strains PA721B-39 and PA824.

To test the effect of increasing serA expression on pantothenate production, PA1010 and PA1013 were grown in test tube cultures of SVY glucose plus 5 g/L β-alanine at 43° C. for 48 hours. As shown by the data presented in Table 7, PA1010 produced on average more pantothenate (4.7 g/L) than its parent strain PA721B-39 (4.1 g/L). Similarly, PA1013 produced on average more pantothenate (4.1 g/L) than its parent strain PA824 (3.1 g/L).

TABLE 7
Production of pantothenate and HMBPA by PA1010 and PA1013
compared to PA721B-39 and PA824.

| | | Pantothenate | HMBPA |
|---|---|---|---|
| Strain | OD₆₀₀ | g/L | g/L |
| PA1010 #3 | 16 | 4.8 | 0.23 |
| PA1010 #5 | 15 | 4.5 | 0.26 |
| PA1010 #6 | 22 | 4.7 | 0.24 |
| PA721B-39 | 18 | 4.0 | 0.22 |
| PA721B-39 | 16 | 4.1 | 0.25 |
| PA1013 #2 | 14 | 3.3 | 0.25 |
| PA1013 #4 | 14 | 4.2 | 0.28 |
| PA1013 #5 | 16 | 5.5 | 0.37 |
| PA1013 #8 | 13 | 3.6 | 0.24 |
| PA824 | 17 | 3.0 | 0.27 |
| PA824 | 16 | 3.1 | 0.29 |

### Example V

### Shake Flask and Fermentor Experiments with Strains with Increased Expression of serA and glyA

Based on performance in test tubes, two strains with an amplifiable serA cassette and two strains with an amplifiable glyA cassette were selected, one each from two parents, PA824 and PA721B-39. The four strains were grown beside the parents in shake flasks (Table 8). In Soy flour MOPS Glucose (SMG) medium, all of the 4 strains produced more pantothenate than their parent strains. In Soy flour MOPS Maltose (SMM) medium one out of the four strains appeared superior to the parent strain.

The serA overexpressing strain and the glyA overexpressing strain from each parent were run simultaneously in 10-liter Chemap bench fermentors. The glyA overexpressing strain derived from PA824, PA1014-3, that had given the highest pantothenate titer in SMM, also performed the best in fermentors (Table 9). Strain PA1014-3 produced 71 g/l pantothenate in 36 hours in the culture supernatant and 86 g/l pantothenate in 48 hours in the culture supernatant compared to the parent PA824 which produced 41 g/l and 46 g/l pantothenate, respectively. The serA strain, PA1012-4, also produced significantly more pantothenate than the PA824 control in the culture supernatant, 52 g/l and 60 g/l at 36 and 48 hours, respectively. These results clearly demonstrate the effectiveness of increasing both glyA and serA.

The serA overexpressing and glyA overexpressing derivatives of PA721B-39 were clearly improved over their parent strain as well. Both produced about 80 g/l pantothenate (82 g/l and 79 g/l, respectively) in the culture supernatants in 48 hours. The effect of the increased PanB levels in the PA721B-39 derivatives versus the PA824 derivatives manifests itself in the reduction of HMBPA. PA721B-39 and its derivatives produce less HMBPA after 48 hours than PA824 or even PA668-24. Increasing GlyA also appears to lower the flow of carbon to HMBPA.

TABLE 8
Shake flask evaluation of pantothenate production strains overexpressing
ser A or gly A.

| Carbon | | Added | HMBPA | Pantothenate |
|---|---|---|---|---|
| source | Strain | cassette | (g/l) | (g/l) |
| Glucose | PA824 | | 3.5 | 4.0 |
| | PA1012-4 | serA | 3.0 | 4.6 |
| | PA1014-3 | gly A | 2.5 | 4.7 |
| | PA721B-39 | | 0.9 | 5.0 |
| | PA1010-6 | serA | 1.9 | 9.6 |
| | PA1011-2 | gly A | 1.7 | 10.0 |
| Maltose | PA824 | | 1.2 | 10.4 |
| | PA1012-4 | serA | 0.8 | 9.8 |
| | PA1014-3 | gly A | 1.1 | 16.1 |
| | PA721B-39 | | 0.6 | 11.6 |
| | PA1010-6 | serA | 0.5 | 10.2 |
| | PA1011-2 | gly A | 0 | 10.3 |

All data are the average of duplicate shake flasks after 48 hours.
Conditions: 40 ml medium/200 ml baffled shake flask, 4× Bioshield covers, 300 rpm, 2.5% inoculum and 43° C.
Medium: 20 g/l Cargill 200/20 soy flour, 1×PSTE, 8 g/l (NH4)2SO4 and 5g/l glutamate.
Buffer: 0.1M phosphate pH 72 and 0.3M MOPS pH 7.2.

Carbon Source (Sterilized separately as 20× stock): 60 g/l glucose or maltose w/10 mM Mg and 1.4 mM Ca.

TABLE 9
10 liter fermentor evaluations of pantothenate production strains
overexpressing serA or glyA.

| | | | | HMBPA | | Pantothenate | |
|---|---|---|---|---|---|---|---|
| | | | | (g/l) | | (g/l) | |
| | | | Added | 36 | | 36 | |
| run | Strain | Parent | cassette | hrs | 48 hrs | hrs | 48 hrs |
| P285 | PA824 | | | 18 | 25 | 41 | 46 |
| P284 | PA1012-4 | PA824 | serA | 20 | 21 | 52 | 60 |
| P286 | PA1014-3 | PA824 | glyA | 14 | 16 | 71 | 86 |
| P259 | PA721B-39 | | | 4 | 5 | 34 | 42 |
| P287 | PA1010-6 | PA721B- | serA | 4 | 5 | 65 | 82 |
| | | 39 | | | | | |
| P289 | PA1011-2 | PA721B- | glyA | 2 | 3 | 56 | 79 |
| | | 39 | | | | | |
| P275 | PA668-24 | PA824 | | 3 | 9 | 55 | 72 |

The medium used is PFM-222. It is the same as medium PFM-155 described in U.S. Ser. No. 60/262,995 (filed Jan. 19, 2001except for the following changes: (1) In the Batch Material: There is no Amberex 1003. Cargill 200/20 (soy flour) 40 g/L has been changed to Cargill 20-80 (soy grits) 50 g/L, MgSO₄.7H₂O is replaced with MgCl₂.7H₂0, 1 g/L, and SM-1000X is replaced with PSTE-1000X (PSTE-1000X=MnCl₂.4H₂O, 2.0 g/L; ZnSO₄.7H₂O, 1.5 g/L; CoCl₂.6H₂O, 2.0 g/L; CuSO₄.5H₂O, 0.25 g/L; Na₂MoO₄.2H₂O, 0.75 g/L). In the Feed Materials: SM-1000X is replaced with PSTE-1000X

Increasing pantothenate production can also be achieved by combining overexpression of serA and glyA in a single strain, and/or by introducing a mutation that leads to feedback resistant serA or glyA, or both.

### Example VI

### Increasing the Expression of the glyA Gene by Mutating the purR Gene

As described in Examples III and V, expression of the glyA gene can be increased by adding one or more copies of a cassette in which the glyA gene is driven by a strong, constitutive promoter. An alternative method to increase glyA expression is to alter its regulation. Literature describing a glyA::lacZ fusion suggests that the glyA promoter is of moderate strength under normal conditions (about 400 Miller Units), but that this promoter is capable of being induced to relatively high levels (1,800 Miller units) if its negative regulator, the purR gene, is deleted (Saxild et al. (2001) J. Bacteriol. 183:6175-6183). Therefore, experiments were preformed to determine if glyA expression, and consequently pantothenate production, could be increased by deleting purR from a pantothenate production strain.

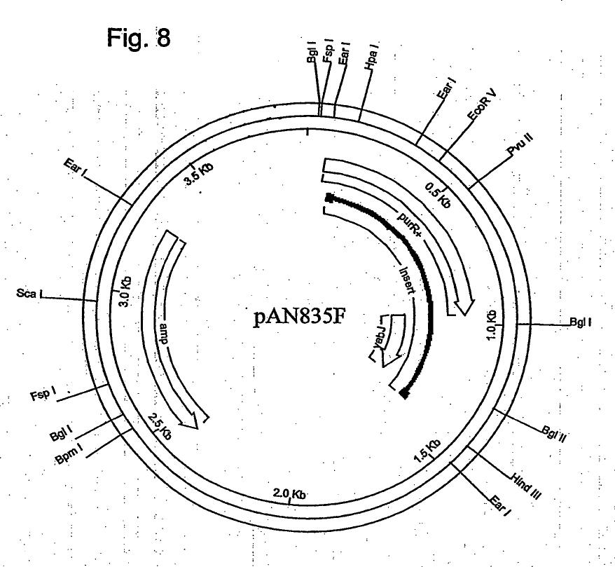

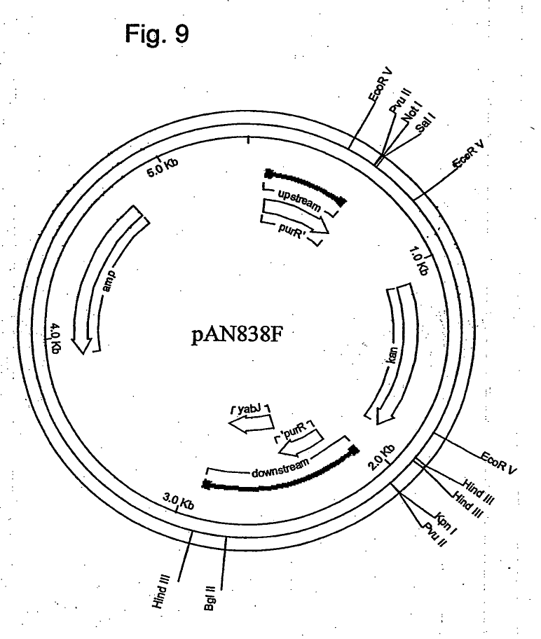

The B. subtilis purR gene was amplified from PY79 chromosomal DNA by PCR, and the resulting fragment was cloned into PvuII cleaved pGEM5-Zf(+) vector DNA to give plasmid pAN835F (SEQ ID NO:26, FIG. 8). This step eliminated the PvuII sites at both ends of the insert, leaving a unique PvuII site in the middle of the purR open reading frame. Next, a blunt PCR DNA fragment containing the Gram positive kanamycin resistance gene from pAN363F (SEQ ID NO:27) was ligated into this unique PvuII site of pAN835F to give pAN838F (SEQ ID NO:28, FIG. 9).

pAN838F was then transformed into PY79, PA668-24, and PA824, selecting for kanamycin resistance at 10 mg/l to give new sets of strains named PA1059, PA1060, and PA1061, respectively. It was shown by PCR that all new isolates contained the disrupted purR::kan allele that was expected from a double crossover event. Several isolates of PA1060 and PA1061 were tested for pantothenate production in test tube cultures grown in SVY glucose plus β-alanine (Table 9). The best isolates derived from PA668-24, PA1060-2 and PA1060-4, gave an improvement from 3.0 g/l pantothenate to 5.3 to 5.1 g/l, respectively, which is an increase of 75%. Likewise, the best isolates derived from PA824, PA1061-1 and PA1061-2,gave an increase from about 3.1 g/l to 5.4 g/l, also a 75% gain. These results suggest that the glyA gene is substantially induced in these new strains by disruption of the purR gene. Alternatively, the improvements in pantothenate production in PA1060 and-PA1061 may be due to more complex pleiotropic effects. In either case, deregulation of the purR regulon has a positive effect on pantothenate production.

In other embodiments, the purR disruption can be installed in other pantothenate production strains, for example those that have an integrated P₂₆serA allele or more than one copy of the P₂₆panBCD operon. The purR gene can also be used as a site for addition of desired expression cassettes, such as P₂₆panB. One can also use resistance to the guanine analogs, such as 8-azaguanine, as a selection for a purR mutation.

TABLE 10
Production of pantothenate and fantothenate by derivatives of
PA824 and PA668-24 containing disrupted purR, in test
tube cultures grown in SVY glucose plus 5 g/l β-alanine.

| | | | new | | [fan] | [pan] |
|---|---|---|---|---|---|---|
| Strain | inoculum* | parent | feature | OD₆₀₀ | g/l | g/l |
| PA668-24 | cam 5, tet 7.5 | PA824 | — | 9 | b.d. | 3.0 |
| ″ | ″ | ″ | — | 12 | b.d. | 3.0 |
| PA1060-1 | cam 5, tet 7.5 | PA668- | purR::kan | 14 | 0.14 | 4.5 |
| | | 24 | | | | |
| PA1060-2 | ″ | PA668- | ″ | 12 | b.d. | 5.3 |
| | | 24 | | | | |
| PA1060-3 | ″ | PA668- | ″ | 12 | b.d. | 4.5 |
| | | 24 | | | | |
| PA1060-4 | ″ | PA668- | ″ | 16 | 0.11 | 5.1 |
| | | 24 | | | | |
| PA824 | tet 30 | PA377 | — | 9 | 0.25 | 3.2 |
| ″ | ″ | ″ | — | 11 | 0.22 | 3.0 |
| PA1061-1 | tet 15 | PA824 | purR::kan | 13 | 0.45 | 5.4 |
| PA1061-3 | ″ | ″ | ″ | 14 | 0.39 | 5.4 |
| PA1061-4 | ″ | ″ | ″ | 11 | 0.40 | 4.7 |

b.d. = below detection
*Concentration of antibiotics in the petri plate from which the inoculum colony was taken.

### Example VI

### Overexpression of the serA Gene from a Non-amplifiable Cassette

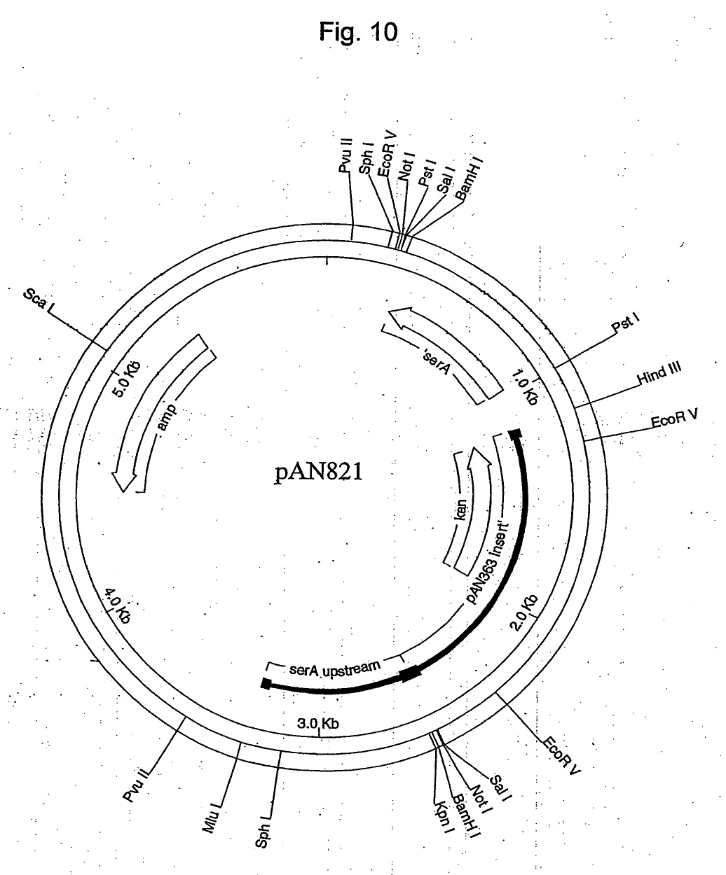

This Example describes another method to increase serine production, in which a two step procedure deposits a strong, constitutive promoter (P₂₆) in front of the chromosomal serA gene. Two plasmids were constructed, each containing about 700 base pairs of DNA sequence from the region immediately upstream of the native serA gene. The first plasmid, pAN821, also contains the 3′ half of the serA coding region, and in between the two aforementioned sequences, a kanamycin resistance gene (SEQ ID NO:30, FIG. 10). When transformed into B. subtilis, selecting for kanamycin resistance, pAN821 will give a disruption of the serA gene, leading to serine auxotrophy. This creates a genetic sequence termed the ΔserA::kan allele.

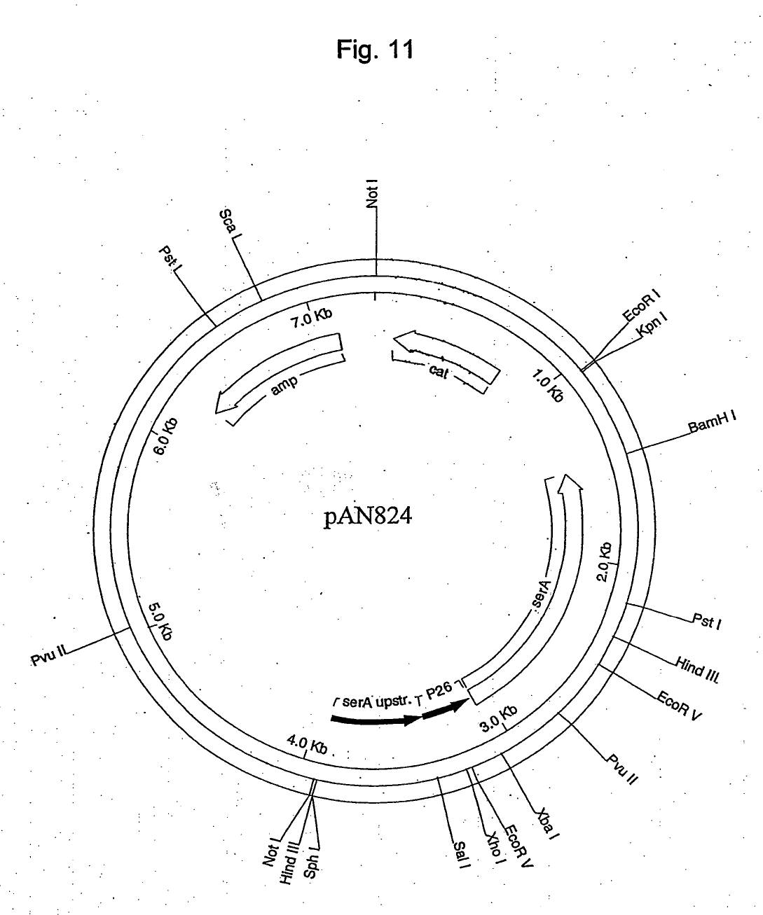

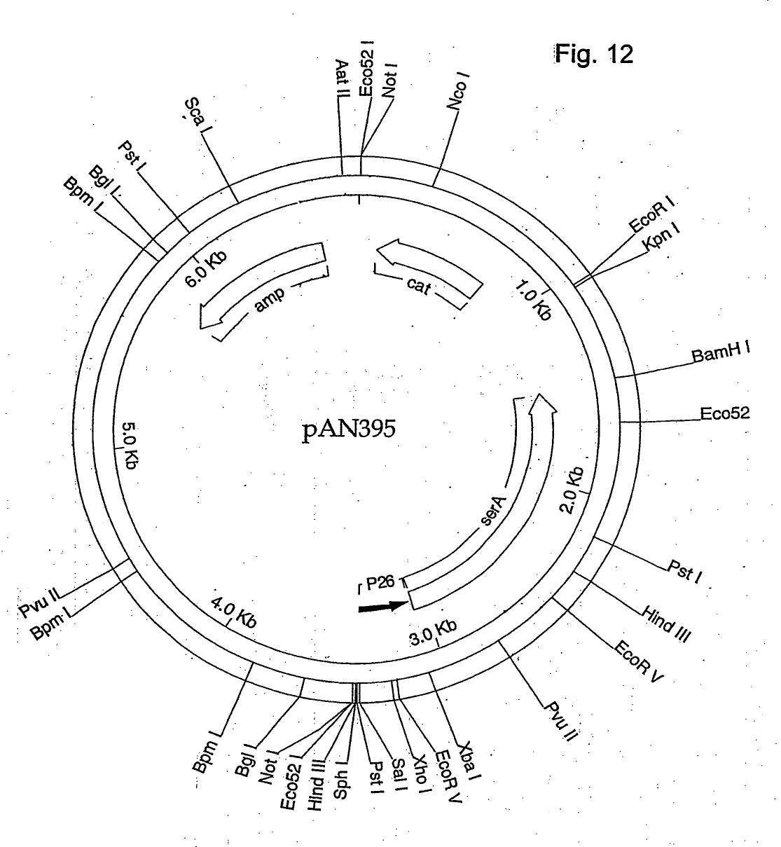

The second plasmid, designed to introduce the P₂₆ serA structure, was constructed by inserting the serA upstream sequence at the 5′ end of the P₂₆ promoter in pAN395. The resulting plasmid, pAN824, is shown in FIG. 11 (SEQ ID NO:31). The plasmid pAN395 is similar to pAN393 described in Example IV. The open reading frame of the serA gene was synthesized by PCR using B. subtilis PY79 DNA as the template. The upstream primer contains an XbaI site and a moderately strong synthetic ribosome binding site, RBS2. The downstream primer contains a BamHI site. This serA open reading frame was used to replace the panBCD genes in the medium copy plasmid, pAN006, to give pAN395 (SEQ ID NO:29, FIG. 12). This plasmid contains the serA gene expressed from the P₂₆ promoter and the RBS2 ribosome binding site.

The ΔserA::kan allele from pAN821 was introduced into strain PA824 to give PA1026. As expected, PA1026 did not grow on minimal medium. In the second step, the P₂₆ serA cassette from plasmid pAN824 was introduced into PA1026, selecting for serine prototrophy, to give strain PA1028. Several PA1028 isolates were confirmed to have the expected chromosomal structure (P₂₆ serA) by diagnostic PCR. These isolates were then tested for pantothenate production in test tube cultures grown for 48 hours in SVY plus 5 g/l β-alanine (Table 11). The PA1028 isolates (derived from PA824) gave increases from 10% to 25% in pantothenate production. As shown in Table 12, in shake flask experiments, PA824 produced about 7 g/l pantothenate, whereas PA1028 produced 11 g/l.

### Example VIII

### Construction of Pantothenate Producing Strains that Contain Both an Integrated Non-amplifiable P₂₆ serA Cassette and an Amplifiable P₂₆ glyA Cassette

Since a non-amplifiable P₂₆ serA cassette integrated at serA led to higher pantothenate synthesis (see, e.g., Table 12), and since a chloramphenicol amplifiable P₂₆ glyA cassette at glyA led to much higher pantothenate synthesis (see, e.g., PA1014-3, Table 8), it was proposed that a combination of the two might be synergistic. Strain PA1028-4, which is the derivative of PA824 that contains the non-amplifiable P₂₆ serA cassette integrated at serA, was transformed to chloramphenicol resistance at 5 mg/l using chromosomal DNA from PA1014-3, to give a set of strains named PA1038, which now contain the chloramphenicol amplifiable P₂₆ glyA cassette. PA1038 isolates were tested for pantothenate production using standard test tube cultures grown in SVY plus β-alanine (Table 13). As expected, PA1038 showed a dramatic increase in pantothenate production from about 4.2 g/l by PA824 to 6.6 to 7.5 g/l by the PA1038 set. Isolates PA1038-3 and PA1038-12 were further tested in shake flasks as shown in Table 12. Both produced an average of 13.6 g/l pantothenate, as compared to the 7.4 g/l pantothenate produced by PA824.

TABLE 11
Production of pantothenate and fantothenate by derivatives of
PA824 that contain a single copy of P₂₆ serA at the serA locus,
in 48 hour test tube cultures grown in SVY plus 5 g/l β-alanine.

| Strain | parent | OD₆₀₀ | [fan] g/l | [pan] g/l |
|---|---|---|---|---|
| PA824 | | 17 | 0.44 | 4.0 |
| PA824 | | 15 | 0.45 | 4.0 |
| PA1028-1 | PA824 | 13 | 0.46 | 4.4 |
| PA1028-2 | ″ | 18 | 0.49 | 4.9 |
| PA1028-3 | ″ | 15 | 0.44 | 4.4 |
| PA1028-4 | ″ | 13 | 0.43 | 4.5 |
| PA1028-5 | ″ | 14 | 0.45 | 4.4 |
| PA1028-6 | ″ | 11 | 0.43 | 4.8 |
| PA1028-8 | ″ | 15 | 0.51 | 5.0 |

TABLE 12
Shake flask evaluation of pantothenate production
strains overexpressing serA and/or glyA.

| | | glyA | serA | Fantothenate | Pantothenate |
|---|---|---|---|---|---|
| Strain | Parent | cassette | cassette | (g/l) | (g/l) |
| PA824 | | | | 0.6 | 7.4 |
| PA1014-3 | PA824 | N × P₂₆glyA | | 0.7 | 12.0 |
| PA1028-4 | PA824 | | P₂₆serA @ serA | 0.8 | 11.1 |
| PA1038-3 | PA1028-4 | N × P₂₆glyA | P₂₆serA @ serA | 0.5 | 13.6 |
| PA1038-12 | PA1028-4 | N × P₂₆glyA | P₂₆serA @ serA | 0.6 | 13.6 |

All data are the average of duplicate shake flasks after 48 hours.
Conditions: 40 ml medium/200 ml baffled shake flask, 4× Bioshield covers, 300 rpm, 2.5% inoculum and 43° C.
Inoculum: SVY base w/maltose 24 hours at 43° C.
Medium: 20 g/l Cargill 200/20 soy flour, 8 g/l (NH₄)₂SO₄, 5 g/l glutamate and 1× PSTE.
Buffer: 0.1M phosphate pH 7.2 and 0.3M MOPS pH 7.2.

Carbon Source (Sterilized separately as 20× stock): 30 g/l maltose, 5 mM MgCl₂ and 0.7 mM CaCl₂

TABLE 13
Pantothenate production by PA1038, a derivative of PA824 that
contains a non-amplifiable P₂₆ serA cassette at serA and an
amplifiable P₂₆ glyA cassette at glyA.

| | Inoculum | | | |
|---|---|---|---|---|
| Strain | Medium | OD₆₀₀ | [Fan] g/L | [Pan] g/L |
| PA824 | tet 15 | 16 | 0.56 | 4.4 |
| PA824 | ″ | 14 | 0.59 | 4.3 |
| PA824 | tet 30 | 12 | 0.57 | 4.3 |
| PA824 | ″ | 14 | 0.58 | 4.2 |
| PA1038-3 | cam 5, tet 15 | 16 | 0.47 | 7.2 |
| PA1038-4 | ″ | 14 | 0.49 | 7.0 |
| PA1038-5 | ″ | 15 | 0.52 | 7.0 |
| PA1038-6 | ″ | 15 | 0.51 | 7.2 |
| PA1038-9 | ″ | 14 | 0.56 | 7.2 |
| PA1038-11 | ″ | 13 | 0.49 | 6.6 |
| PA1038-12 | ″ | 16 | 0.58 | 7.5 |

Test tube cultures were grown with SVY glucose plus 5 g/l β-alanine at 43° C. for 48 hours.

### Example IX

### Increasing the Production of MTF by Altering the Glycine Cleavage Pathway

As demonstrated with the above examples, increasing MTF production in bacteria increases the production of pantothenate in strains that have been engineered to produce more pantothenate by manipulation of the panBCD and/or panE genes. It has been demonstrated that pantothenate production can be increased by increasing the expression of the glyA or the serA gene. Stronger promoters or ribosome binding sites can be used to increase glyA or serA expression as demonstrated in Examples III through V and VII through VIII. Alternatively, the expression of the glyA gene can be deregulated in Bacillus by disrupting the purR repressor gene as illustrated in Example VI.

Another method to increase MTF production is to enhance the expression of enzymes of the glycine cleavage pathway. For example, enzymes encoded by the gcvT, gcvPA, gcvPB, gcvH, and pdhD genes catalyze the breakdown of glycine to MTF, CO₂, and NH₃. A strong, constitutive promoter, such as the SP01 phage P₂₆ promoter described previously, can be cloned in front of the gcvT-gcvPA-gcvPB operon or in front of the gcvH or pdhD gene to enhance their expression. In addition to the above mentioned approaches, additional glycine, which is inexpensive, can be added to the medium to further enhance MTF production by any strain engineered as described herein.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A process for the enhanced production of pantothenate, comprising culturing a microorganism having a deregulated methylenetetrahydrofolate (MTF) biosynthetic pathway, under conditions such that pantothenate production is enhanced.

2. A process for the enhanced production of pantothenate, comprising culturing a microorganism having
(i) a deregulated pantothenate biosynthetic pathway, and
(ii) a deregulated methylenetetrahydrofolate (MTF) biosynthetic pathway, under conditions such that pantothenate production is enhanced.

3. The process of claim 2, wherein said microorganism has at least two pantothenate biosynthetic enzymes deregulated.

4. The process of claim 2, wherein said microorganism has at least three pantothenate biosynthetic enzymes deregulated.

5. The process of claim 2, wherein said microorganism has at least four pantothenate biosynthetic enzymes deregulated.

6. The process of claim 5, wherein said microorganism has a deregulated ketopantoate hydroxymethyltransferase, a deregualted ketopantoate reductase, a deregulated pantothenate synthetase and a deregulated aspartate-α-decarboxylase.

7. The process of claim 1 or 2, wherein said microorganism further has a deregulated isoleucine-valine (ilv) biosynthetic pathway.

8. The process of claim 7, wherein said microorganism has at least two isoleucine-valine (ilv) biosynthetic enzymes deregulated.

9. The process of claim 7, wherein said microorganism has at least three isoleucine-valine (ilv) biosynthetic enzymes deregulated.

10. The process of claim 9, wherein said microorganism has a deregulated acetohydroxyacid acid synthetase, a deregulated acetohydroxyacid isomeroreductase, and a deregulated dihydroxyacid dehydratase.

11. The process of any one of claims 1 to 10, wherein the microorganism has at least one MTF biosynthetic enzyme deregulated.

12. The process of claim 11, wherein the microorganism has a deregulated glyA gene.

13. The process of claim 11, wherein the microorganism has a deregulated serA gene.

14. The process of claim 11, wherein the microorganism has a deregulated glyA gene and a deregulated serA gene.

15. The process of claim 12 or 14, wherein the microorganism has a mutated, deleted or disrupted purR gene.

16. A process for the enhanced production pantothenate, comprising culturing a microorganism having a deregualted pantothenate biosynthetic pathway, a deregulated isoleucine-valine (ilv) biosynthetic pathway, and a deregulated methylenetetrahydrofolate (MTF) biosynthetic pathway deregulated, such that production of pantothenate is enhanced.

17. A process for the production pantothenate, comprising culturing a microorganism having a deregulated pantothenate biosynthetic pathway, a deregulated isoleucine-valine (ilv) biosynthetic pathway, and a deregulated methylenetetrahydrofolate (MTF) biosynthetic pathway, such that at least 50 g/L pantothenate is produced after 36 hours of culturing the microorganism.

18. The process of claim 17, comprising culturing the microorganism such that at least 60 g/L pantothenate is produced after 36 hours of culturing the microorganism.

19. The process of claim 17, comprising culturing the microorganism such that at least 70 g/L pantothenate is produced after 36 hours of culturing the microorganism.

20. A process for the production pantothenate, comprising culturing a microorganism having a deregulated pantothenate biosynthetic pathway, a deregulated isoleucine-valine (ilv) biosynthetic pathway, and a deregulated methylenetetrahydrofolate (MTF) biosynthetic pathway deregulated, such that at least 60 g/L pantothenate is produced after 48 hours of culturing the microorganism.

21. The process of claim 20, comprising culturing the microorganism such that at least 70 g/L pantothenate is produced after 48 hours of culturing the microorganism.

22. The process of claim 20, comprising culturing the microorganism such that at least 80 g/L pantothenate is produced after 48 hours of culturing the microorganism.

23. The process of any one of the preceding claims, wherein pantothenate production is further enhanced by regulating pantothenate kinase activity.

24. The process of claim 23, wherein pantothenate kinase activity is decreased.

25. The process of claim 24, wherein CoaA is deleted and CoaX is downregulated.

26. The process of claim 24, wherein CoaX is deleted and CoaA is downregulated.

27. The process of claim 24, wherein CoaX and CoaA are downregulated.

28. The process of any one of the above claims, wherein said microorganism is cultured under conditions of excess serine.

29. A process for producing pantothenate comprising culturing a microorganism having a deregulated pantothenate biosynthetic pathway under conditions of excess serine, such that pantothenate in produced.

30. The process of any one of the above claims, wherein said microorganism has the pantothenate biosynthetic pathway deregulated such that pantothenate production is independent of β-alanine feed.

31. The process of any one of the above claims wherein the microorganism is a Gram positive microorganism.

32. The process of any one of the above claims wherein the microorganism belongs to the genus Bacillus.

33. The process of any one of the above claims, wherein the microorganism is Bacillus subtilis.

34. A product synthesized according to the process of any one of the above claims.

35. A composition comprising pantothenate produced according to the process of any one of the above claims.

36. A recombinant microorganism for the enhanced production of pantothenate, said microorganism having a deregulated pantothenate biosynthetic pathway, and a deregulated methylenetetrahydrofolate (MTF) biosynthetic pathway.

37. A recombinant microorganism for the enhanced production of pantothenate, said microorganism having a deregulated pantothenate biosynthetic pathway, a deregulated methylenetetrahydrofolate (MTF) biosynthetic pathway, and a deregulated isoleucine-valine (ilv) pathway.

38. The microorganism of claim 36 or 37, further having reduced pantothenate kinase activity.

39. The microorganism of any one of claims 36-38 which is a Gram positive microorganism.

40. The microorganism of any one of claims 36-38 belonging to the genus Bacillus.

41. The microorganism of any one of claims 36-38 which is Bacillus subtilis.

42. A process for producing pantothenate comprising culturing a recombinant microorganism having:
(a) a deregulated panB gene;
(b) a deregulated panD gene; and
(c) at least one deregulated isoleucine-valine (ilv) biosynthetic enzyme-encoding gene; under conditions such that at least 30 g/l pantothenate is produced after 36 hours of culturing the microorganism.

43. The process of claim 42, wherein said microorganism further has a deregulated methylenetetrahydrofolate (MTF) biosynthetic pathway and said microorganism is cultured under conditions such that at least 50 g/l pantothenate is produced after 36 hours of culturing the microorganism.

44. A process for producing pantothenate comprising culturing a recombinant microorganism having:
(a) a deregulated panB gene; and
(b) a deregulated panD gene;
under conditions of excess serine, such that at least 50 g/l pantothenate is produced after 36 hours of culturing the microorganism.

45. A process for producing pantothenate comprising culturing a recombinant microorganism having:
(a) a deregulated panB gene;
(b) a deregulated panD gene; and
(c) a deregulated methylenetetrahydrofolate (MTF) biosynthetic pathway; under conditions of excess valine, such that at least 50 g/l pantothenate is produced after 36 hours of culturing the microorganism.

46. A process for producing pantothenate comprising culturing a recombinant microorganism having:
(a) a deregulated panB gene;
(b) a deregulated panD gene; and
(c) a deregulated glyA gene;
under conditions of excess valine, such that at least 50 g/l pantothenate is produced after 36 hours of culturing the microorganism.

47. A process for producing pantothenate comprising culturing a recombinant microorganism having:
(a) a deregulated panB gene;
(b) a deregulated panD gene; and
(c) a mutated, deleted or disrupted purR gene;
under conditions of excess valine, such that at least 50 g/l pantothenate is produced after 36 hours of culturing the microorganism.

48. A process for producing pantothenate comprising culturing a recombinant microorganism having:
(a) a deregulated panB gene;
(b) a deregulated panD gene; and
(c) a deregulated serA gene;
under conditions of excess valine, such that at least 50 g/l pantothenate is produced after 36 hours of culturing the microorganism.

49. A process for producing pantothenate comprising culturing a recombinant microorganism having:
(a) a deregulated panB gene;
(b) a deregulated panD gene;
(c) a deregulated serA gene;
(d) a deregulated glyA gene; and
under conditions of excess valine, such that at least 50 g/l pantothenate is produced after 36 hours of culturing the microorganism.


SEQUENCE LISTING (31 sequences)

SEQ ID NO: 1 - DNA, 194 bp, Artificial Sequence
  Description of Artificial Sequencepromoter sequence
gctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgt

SEQ ID NO: 2 - DNA, 163 bp, Artificial Sequence
  Description of Artificial Sequencepromoter sequence
gcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgc

SEQ ID NO: 3 - DNA, 127 bp, Artificial Sequence
  Description of Artificial Sequencepromoter sequence
gaggaatcatagaattttgtcaaaataattttattgacaacgtcttattaacgttgatataatttaaattttatttgacaaaaatgggctcgtgttgtacaataaatgtagtgaggtggatgcaatg

SEQ ID NO: 4 - DNA, 24 bp, Artificial Sequence
  Description of Artificial Sequenceribosome binding site
taaacatgaggaggagaaaacatg

SEQ ID NO: 5 - DNA, 28 bp, Artificial Sequence
  Description of Artificial Sequenceribosome binding site
attcgagaaatggagagaatataatatg

SEQ ID NO: 6 - DNA, 13 bp, Artificial Sequence
  Description of Artificial Sequenceribosome binding site
agaaaggaggtga

SEQ ID NO: 7 - DNA, 23 bp, Artificial Sequence
  Description of Artificial Sequenceribosome binding site
ttaagaaaggaggtgannnnatg

SEQ ID NO: 8 - DNA, 23 bp, Artificial Sequence
  Description of Artificial Sequenceribosome binding site
ttagaaaggaggtgannnnnatg

SEQ ID NO: 9 - DNA, 23 bp, Artificial Sequence
  Description of Artificial Sequenceribosome binding site
agaaaggaggtgannnnnnnatg

SEQ ID NO: 10 - DNA, 22 bp, Artificial Sequence
  Description of Artificial Sequenceribosome binding site
agaaaggaggtgannnnnnatg

SEQ ID NO: 11 - DNA, 25 bp, Artificial Sequence
  Description of Artificial Sequenceribosome binding site
ccctctagaaggaggagaaaacatg

SEQ ID NO: 12 - DNA, 24 bp, Artificial Sequence
  Description of Artificial Sequenceribosome binding site
ccctctagaggaggagaaaacatg

SEQ ID NO: 13 - DNA, 23 bp, Artificial Sequence
  Description of Artificial Sequenceribosome binding site
ttagaaaggaggatttaaatatg

SEQ ID NO: 14 - DNA, 23 bp, Artificial Sequence
  Description of Artificial Sequenceribosome binding site
ttagaaaggaggtttaattaatg

SEQ ID NO: 15 - DNA, 23 bp, Artificial Sequence
  Description of Artificial Sequenceribosome binding site
ttagaaaggaggtgatttaaatg

SEQ ID NO: 16 - DNA, 23 bp, Artificial Sequence
  Description of Artificial Sequenceribosome binding site
ttagaaaggaggtgtttaaaatg

SEQ ID NO: 17 - DNA, 28 bp, Artificial Sequence
  Description of Artificial Sequenceribosome binding site
attcgagaaaggaggtgaatataatatg

SEQ ID NO: 18 - DNA, 27 bp, Artificial Sequence
  Description of Artificial Sequenceribosome binding site
attcgagaaaggaggtgaataataatg

SEQ ID NO: 19 - DNA, 28 bp, Artificial Sequence
  Description of Artificial Sequenceribosome binding site
attcgtagaaaggaggtgaattaatatg

SEQ ID NO: 20 - DNA, 51 bp, Artificial Sequence
  Description of Artificial Sequence5′ PCR primer
ccctctagaggaggagaaaacatgtttcgagtattggtctcagacaaaatg

SEQ ID NO: 21 - DNA, 43 bp, Artificial Sequence
  Description of Artificial Sequence3′ PCR primer
cccggatccaattatggcagatcaatgagcttcacagacacaa

SEQ ID NO: 22 - DNA, 48 bp, Artificial Sequence
  Description of Artificial Sequence5′ PCR primer
ggatctagaggaggtgtaaacatgaaacatttacctgcgcaagacgaa

SEQ ID NO: 23 - DNA, 43 bp, Artificial Sequence
  Description of Artificial Sequence3′ PCR primer
cggggatcccccatcaacaattacacacttctattgattctac

SEQ ID NO: 24 - DNA, 7926 bp, Artificial Sequence
  Description of Artificial SequenceserA overexpression
gaattttgcggccgcttcgaaagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgaattcggtaccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacaggatccaattatggcagatcaatgagcttcacagacacaatatcagggacatttgttagttctttcacaattttatcttccagatgtctgtcaaaggaaagcatcatgatggcttctccgcctttttccttacggccaacctgcatagttgcaatgttaatatcattatctccgagaatacgtcctactcggccgatgacacctgttgtatcttgatgctggatatacaccaagtgaccagtcggataaaaatcaatattaaatccattgatctcgacaattcgttctccgaaatgaggaatatacgtagccgttacagtaaaggtgctgcggtctcctgtcacttttacgctgatgcagttatcgtatccagattcagaagaggaaattttttcactgaagctaatgccgcgttcttttgcgacacccccggcattgacctcattaacagtagagtctacgcgcggttttaaaaagcctgacagaagggcttttgtaatgaacgatgtttcaagtttagcaattgtgccttcatattgaatggcaacatcctgtactggttctttcatgcactgtgatacaaggctgccaatttttcctgcaatttgatggtaaggcttaattttagcaaattcatcttttgtcatggcaggcaggttgatagctgacatgacaggcaggccttttgcgaactgcagaacttcttctgacacttgggcggcgacattgagctgtgcttctttcgttgatgctcccaagtgaggagtggcaatgactaatggatgatcaacaagtttgttgtcaactggcggttcgacttcgaaaacgtcaagcgctgctcccgcaacatgcccgttttccaaagcttcgagaagtgctgcttcatcgataattccgcctcgcgcacagttaattaagcgaacgccttttttcgtttttgcaatcgtttctttattcaataagccttttgtttcttttgttaaaggcgtgtgaacggtaatgatatccgcactttcaagcacttcttcaaatgtacggctgtttacgccgatttttttcgctctttcttccgttaagaaaggatcaaaaacgtgcacagtcataccgaacgctcctcgacgctgtgcaatttcacttccgattcggcctaatcctacaataccaagcgtttttccataaagctctgaaccgacataagctgtgcggttccactctctggatttcactgagatattagcctgcggaatgtgtctcattaaagaagagatcattgcaaatgtatgctcagctgtcgaaatggtgttgccgttcggagcattgatcacgattaccccgtgtttcgtagcctcatcaatatcgatattatcgacaccgacaccggctcttccgacaatttttaaagaagtcattttgttgaaaaggtcttctgttacttttgtcgcgcttcgcaccaaaagagcatcaaaagtatgtaattcatcttctgcatctgctacgtttttttgaacgatttcaataaagtctgattcaataagtggctgtaaaccgtcgttgctcattttgtctgagaccaatactcgaaacatgttttctcctcctctagagcgtcctgctgttgttaagattattataccacaccttgtagataaagtcaacaactttttgcaaaatttttcaggaattttagcagaggttgttctggatgtagaacaaaacatctttccgctcttgtgctgttaggatatctttcttggaagctaggtaggcctcgagttatggcagttggttaaaaggaaacaaaaagaccgttttcacacaaaacggtctttttcgatttctttttacagtcacagccacttttgcaaaaaccggacagcttcatgccttataactgctgtttcggtcgacaagcttcgcgaagcggccgcaaaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgactatgcttgtaaaccgttttgtgaaaaaatttttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaac

SEQ ID NO: 25 - DNA, 7701 bp, Artificial Sequence
  Description of Artificial SequenceglyA overexpression
gaattttgcggccgcttcgaaagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgaattcggtaccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacaggatcccccatcaacaattacacacttctattgattctacaaaaaaagacattgagtttcaagaacatcgtcaaaaaacccgccgggcataagcccaagcgggttttaggatcttaataatctaattctttatataaaggaaatttatcagtcagagcagctacacgctgtcttgcttcttcaagttttccttcatcttcgtggtttttcaatgcaagcgcaatgatagcaccgacttcttctaatgcgtctccgtcaaaaccgcggctggttacagcagctgtaccaagacggatgccgcttgttacgaaaggtttttcaggatcatatggaatcgcgtttttgttagacgtaataccaatttcatcaagtacatgctccgcaaccttaccagtcagtccgagcgaacgaaggtcaacaaggataaggtggttgtctgttccgcctgaaacgagctggatgccctctttcgttaaggcttcagccagacgtttcgcgtttgaaatgacgttttgtgcatatgttttgaaatcgtcctgcaatacttcaccgaatgaaacagcttttgcggcaataacgtgcatcagagggccgccttgaattccagggaagatcgatttatcaattttcttgccaaactcttcacggcaaaggatcataccgccgcgaggaccgcgaagtgttttatgtgttgttgttgtaacgaaatcagcgtaaggaaccgggtttggatgaaggcctgccgcaacaagtcctgcgatatgtgccatatccaccatgaagtaagcgccgacttcatcagcaatttcacggaatttcttaaagtcgattgtacgaggatacgcacttgctcctgctacgataagcttcggtttatgagcgagggctttttcacgcacgtcatcgtaatcaatatattgagtttctttatctacgccgtactcaacaaagttatattgaacaccgctgaagttgactgggcttccgtgtgttaaatggccgccgtgggagaggttcatcccaagtacagtatcgccttgctccaaaatcgtgaagtacactgccatgtttgcttgtgcgcctgaatgaggctgaacgtttacatgctccgctccaaagatttccttcgcgcggtcacgggcgatatcttcaacgacatcgacgtgctcgcatccgccgtagtagcgtttgcccggatatccttctgcgtacttatttgtcaaaacagatccttgtgcttccataaccgcttcacttacaaagttctcagaagcaatcaattcgatcttagtctgttggcgttcacgctcatttttaatggcgttaaacacttgttcgtcttgcgcaggtaaatgtttcatgtttacacctcctctagagcgtcctgctgttgttaagattattataccacaccttgtagataaagtcaacaactttttgcaaaatttttcaggaattttagcagaggttgttctggatgtagaacaaaacatctttccgctcttgtgctgttaggatatctttcttggaagctaggtaggcctcgagttatggcagttggttaaaaggaaacaaaaagaccgttttcacacaaaacggtctttttcgatttctttttacagtcacagccacttttgcaaaaaccggacagcttcatgccttataactgctgtttcggtcgacaagcttcgcgaagcggccgcaaaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgactatgcttgtaaaccgttttgtgaaaaaatttttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcaac

SEQ ID NO: 26 - DNA, 3888 bp, Artificial Sequence
  plasmid
tgcgccgctacagggcgcgtccattcgccattcaggctgcgcaactgttgggaagggcgatcggtgcgggcctcttcgctattacgccagtttgggggtgagttcatgaagtttcgtcgcagcggcagattggtggacttaacaaattatttgttaacccatccgcacgagttaataccgctaacctttttctctgagcggtatgaatctgcaaaatcatcgatcagtgaagatttaacaattattaaacaaacctttgaacagcaggggattggtactttgcttactgttcccggagctgccggaggcgttaaatatattccgaaaatgaagcaggctgaagctgaagagtttgtgcagacacttggacagtcgctggcaaatcctgagcgtatccttccgggcggttatgtatatttaacggatatcttaggaaagccatctgtactctccaaggtagggaagctgtttgcttccgtgtttgcagagcgcgaaattgatgttgtcatgaccgttgccacgaaaggcatccctcttgcgtacgcagctgcaagctatttgaatgtgcctgttgtgatcgttcgtaaagacaataaggtaacagagggctccacagtcagcattaattacgtttcaggctcctcaaaccgcattcaaacaatgtcacttgcgaaaagaagcatgaaaacgggttcaaacgtactcattattgatgactttatgaaagcaggcggcaccattaatggtatgattaacctgttggatgagtttaacgcaaatgtggcgggaatcggcgtcttagttgaagccgaaggagtagatgaacgtcttgttgacgaatatatgtcacttcttactctttcaaccatcaacatgaaagagaagtccattgaaattcagaatggcaattttctgcgtttttttaaagacaatcttttaaagaatggagagacagaatcatgacaaaagcagtccacacaaaacatgccccagcggcaatcgggccttattcacaagggattatcgtcaacaatatgttttacagctcaggccaaatccctttgactccttcaggcgaaatggtgaatggcgatattaaggagcagactcatcaagtattcagcaatttaaaggcggttctggaagaagcgggtgcttcttttgaaacagttgtaaaagcaactgtatttatcgcggatatggaacagtttgcggaagtaaacgaagtgtacggacaatattttgacactcacaaaccggcgagatcttgtgttgaagtcgcgagactcccgaaggatgcgttagtcgagatcgaagttattgcactggtgaaataataagaaaagtgattctgggagagccgggatcacttttttatttaccttatgcccgaaatgaaagctttatgaccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 27 - DNA, 4606 bp, Artificial Sequence
  Description of Artificial Sequenceplasmid
tgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcgggatgcggccgcgtcgacgtgaaataccgcacagatgcgtaaggagaaaataccgcatcaggcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgatgttttatatcttttctaaaagttcgctagataggggtcccgagcgcctacgaggaatttgtatcgccattcgccattcaggctgcgcaactgttgggaagggcgatcggtgcgggtaccgggatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 28 - DNA, 5399 bp, Artificial Sequence
  Description of Artificial Sequenceplasmid
tgcgccgctacagggcgcgtccattcgccattcaggctgcgcaactgttgggaagggcgatcggtgcgggcctcttcgctattacgccagtttgggggtgagttcatgaagtttcgtcgcagcggcagattggtggacttaacaaattatttgttaacccatccgcacgagttaataccgctaacctttttctctgagcggtatgaatctgcaaaatcatcgatcagtgaagatttaacaattattaaacaaacctttgaacagcaggggattggtactttgcttactgttcccggagctgccggaggcgttaaatatattccgaaaatgaagcaggctgaagctgaagagtttgtgcagacacttggacagtcgctggcaaatcctgagcgtatccttccgggcggttatgtatatttaacggatatcttaggaaagccatctgtactctccaaggtagggaagctgtttgcttccgtgtttgcagagcgcgaaattgatgttgtcatgaccgttgccacgaaaggcatccctcttgcgtacgcagctgcggccgcgtcgacaaacccagtgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgatgttttatatcttttctaaaagttcgctagataggggtcccgagcgcctacgaggaatttgtatcaccaggtaccagctgcaagctatttgaatgtgcctgttgtgatcgttcgtaaagacaataaggtaacagagggctccacagtcagcattaattacgtttcaggctcctcaaaccgcattcaaacaatgtcacttgcgaaaagaagcatgaaaacgggttcaaacgtactcattattgatgactttatgaaagcaggcggcaccattaatggtatgattaacctgttggatgagtttaacgcaaatgtggcgggaatcggcgtcttagttgaagccgaaggagtagatgaacgtcttgttgacgaatatatgtcacttcttactctttcaaccatcaacatgaaagagaagtccattgaaattcagaatggcaattttctgcgtttttttaaagacaatcttttaaagaatggagagacagaatcatgacaaaagcagtccacacaaaacatgccccagcggcaatcgggccttattcacaagggattatcgtcaacaatatgttttacagctcaggccaaatccctttgactccttcaggcgaaatggtgaatggcgatattaaggagcagactcatcaagtattcagcaatttaaaggcggttctggaagaagcgggtgcttcttttgaaacagttgtaaaagcaactgtatttatcgcggatatggaacagtttgcggaagtaaacgaagtgtacggacaatattttgacactcacaaaccggcgagatcttgtgttgaagtcgcgagactcccgaaggatgcgttagtcgagatcgaagttattgcactggtgaaataataagaaaagtgattctgggagagccgggatcacttttttatttaccttatgcccgaaatgaaagctttatgaccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 29 - DNA, 6805 bp, Artificial Sequence
  Description of Artificial Sequenceplasmid
ttgcggccgcttcgaaagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgaattcggtaccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacaggatccaattatggcagatcaatgagcttcacagacacaatatcagggacatttgttagttctttcacaattttatcttccagatgtctgtcaaaggaaagcatcatgatggcttctccgcctttttccttacggccaacctgcatagttgcaatgttaatatcattatctccgagaatacgtcctactcggccgatgacacctgttgtatcttgatgctggatatacaccaagtgaccagtcggataaaaatcaatattaaatccattgatctcgacaattcgttctccgaaatgaggaatatacgtagccgttacagtaaaggtgctgcggtctcctgtcacttttacgctgatgcagttatcgtatccagattcagaagaggaaattttttcactgaagctaatgccgcgttcttttgcgacacccccggcattgacctcattaacagtagagtctacgcgcggttttaaaaagcctgacagaagggcttttgtaatgaacgatgtttcaagtttagcaattgtgccttcatattgaatggcaacatcctgtactggttctttcatgcactgtgatacaaggctgccaatttttcctgcaatttgatggtaaggcttaattttagcaaattcatcttttgtcatggcaggcaggttgatagctgacatgacaggcaggccttttgcgaactgcagaacttcttctgacacttgggcggcgacattgagctgtgcttctttcgttgatgctcccaagtgaggagtggcaatgactaatggatgatcaacaagtttgttgtcaactggcggttcgacttcgaaaacgtcaagcgctgctcccgcaacatgcccgttttccaaagcttcgagaagtgctgcttcatcgataattccgcctcgcgcacagttaattaagcgaacgccttttttcgtttttgcaatcgtttctttattcaataagccttttgtttcttttgttaaaggcgtgtgaacggtaatgatatccgcactttcaagcacttcttcaaatgtacggctgtttacgccgatttttttcgctctttcttccgttaagaaaggatcaaaaacgtgcacagtcataccgaacgctcctcgacgctgtgcaatttcacttccgattcggcctaatcctacaataccaagcgtttttccataaagctctgaaccgacataagctgtgcggttccactctctggatttcactgagatattagcctgcggaatgtgtctcattaaagaagagatcattgcaaatgtatgctcagctgtcgaaatggtgttgccgttcggagcattgatcacgattaccccgtgtttcgtagcctcatcaatatcgatattatcgacaccgacaccggctcttccgacaatttttaaagaagtcattttgttgaaaaggtcttctgttacttttgtcgcgcttcgcaccaaaagagcatcaaaagtatgtaattcatcttctgcatctgctacgtttttttgaacgatttcaataaagtctgattcaataagtggctgtaaaccgtcgttgctcattttgtctgagaccaatactcgaaacatgttttctcctcctctagagcgtcctgctgttgttaagattattataccacaccttgtagataaagtcaacaactttttgcaaaatttttcaggaattttagcagaggttgttctggatgtagaacaaaacatctttccgctcttgtgctgttaggatatctttcttggaagctaggtaggcctcgagttatggcagttggttaaaaggaaacaaaaagaccgttttcacacaaaacggtctttttcgatttctttttacagtcacagccacttttgcaaaaaccggacagcttcatgccttataactgctgtttcggtcgacctgcaggcatgcaagcttcgcgaagcggccgccgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 30 - DNA, 5983 bp, Artificial Sequence
  Description of Artificial Sequenceplasmid
tgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcgggatatcactagtgcggccgcctgcaggtcgaccatatgggagagcccggatccaattatggcagatcaatgagcttcacagacacaatatcagggacatttgttagttctttcacaattttatcttccagatgtctgtcaaaggaaagcatcatgatggcttctccgcctttttccttacggccaacctgcatagttgcaatgttaatatcattatctccgagaatacgtcctactcggccgatgacacctgttgtatcttgatgctggatatacaccaagtgaccagtcggataaaaatcaatattaaatccattgatctcgacaattcgttctccgaaatgaggaatatacgtagccgttacagtaaaggtgctgcggtctcctgtcacttttacgctgatgcagttatcgtatccagattcagaagaggaaattttttcactgaagctaatgccgcgttcttttgcgacacccccggcattgacctcattaacagtagagtctacgcgcggttttaaaaagcctgacagaagggcttttgtaatgaacgatgtttcaagtttagcaattgtgccttcatattgaatggcaacatcctgtactggttctttcatgcactgtgatacaaggctgccaatttttcctgcaatttgatggtaaggcttaattttagcaaattcatcttttgtcatggcaggcaggttgatagctgacatgacaggcaggccttttgcgaactgcagaacttcttctgacacttgggcggcgacattgagctgtgcttctttcgttgatgctcccaagtgaggagtggcaatgactaatggatgatcaacaagtttgttgtcaactggcggttcgacttcgaaaacgtcaagcgctgctcccgcaacatgcccgttttccaaagctttttagacatctaaatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttgaaagttggcgtataacatagtatcgacggagccgattttgaaaccacaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcctgatgcggtattttctccttacgcatctgtgcggtatttcacgtcgacgcggccgccatggccgcgggatcccggtaccgaaacatcgttagatttcctcctaaattgacaaactaaatatctgataatttaacatattctcaaaagagtgtcaacgtgtattgacgcagtaaaggataaaagtaaagcctaataaatcaatgatctgacagcttgcaggtaatatatttaatttgaagcaattctctatacagccaaccagttatcgtttataatgtaattaaatttcatatgatcaatcttcggggcagggtgaaattccctaccggcggtgatgagccaatggctctaagcccgcgagctgtctttacagcaggattcggtgagattccggagccgacagtacagtctggatgggagaagatggaggttcataagcgttttgaaattgaatttttcaaacgtttctttgcctagcctaattttcgaaaccccgcttttatatatgaagcggtttttttattggctggaaaagaacctttccgttttcgagtaagatgtgatcgaaaaggagagaatgaagtgaaagtaaaaaaattagttgtggtcagcatgctgagcagcattgcatttgttttgatgctgttaaatttcccgtttccgggtcttccggattatttaaaaatcgattttagcgacgttcccgcaattattgccattctgatttacggacctttggcgggatcactagagggctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 31 - DNA, 7330 bp, Artificial Sequence
  Description of Artificial Sequenceplasmid
ttgcggccgcttcgaaagctgtaatataaaaaccttcttcaactaacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgaattcggtaccaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacaggatccaattatggcagatcaatgagcttcacagacacaatatcagggacatttgttagttctttcacaattttatcttccagatgtctgtcaaaggaaagcatcatgatggcttctccgcctttttccttacggccaacctgcatagttgcaatgttaatatcattatctccgagaatacgtcctactcggccgatgacacctgttgtatcttgatgctggatatacaccaagtgaccagtcggataaaaatcaatattaaatccattgatctcgacaattcgttctccgaaatgaggaatatacgtagccgttacagtaaaggtgctgcggtctcctgtcacttttacgctgatgcagttatcgtatccagattcagaagaggaaattttttcactgaagctaatgccgcgttcttttgcgacacccccggcattgacctcattaacagtagagtctacgcgcggttttaaaaagcctgacagaagggcttttgtaatgaacgatgtttcaagtttagcaattgtgccttcatattgaatggcaacatcctgtactggttctttcatgcactgtgatacaaggctgccaatttttcctgcaatttgatggtaaggcttaattttagcaaattcatcttttgtcatggcaggcaggttgatagctgacatgacaggcaggccttttgcgaactgcagaacttcttctgacacttgggcggcgacattgagctgtgcttctttcgttgatgctcccaagtgaggagtggcaatgactaatggatgatcaacaagtttgttgtcaactggcggttcgacttcgaaaacgtcaagcgctgctcccgcaacatgcccgttttccaaagcttcgagaagtgctgcttcatcgataattccgcctcgcgcacagttaattaagcgaacgccttttttcgtttttgcaatcgtttctttattcaataagccttttgtttcttttgttaaaggcgtgtgaacggtaatgatatccgcactttcaagcacttcttcaaatgtacggctgtttacgccgatttttttcgctctttcttccgttaagaaaggatcaaaaacgtgcacagtcataccgaacgctcctcgacgctgtgcaatttcacttccgattcggcctaatcctacaataccaagcgtttttccataaagctctgaaccgacataagctgtgcggttccactctctggatttcactgagatattagcctgcggaatgtgtctcattaaagaagagatcattgcaaatgtatgctcagctgtcgaaatggtgttgccgttcggagcattgatcacgattaccccgtgtttcgtagcctcatcaatatcgatattatcgacaccgacaccggctcttccgacaatttttaaagaagtcattttgttgaaaaggtcttctgttacttttgtcgcgcttcgcaccaaaagagcatcaaaagtatgtaattcatcttctgcatctgctacgtttttttgaacgatttcaataaagtctgattcaataagtggctgtaaaccgtcgttgctcattttgtctgagaccaatactcgaaacatgttttctcctcctctagagcgtcctgctgttgttaagattattataccacaccttgtagataaagtcaacaactttttgcaaaatttttcaggaattttagcagaggttgttctggatgtagaacaaaacatctttccgctcttgtgctgttaggatatctttcttggaagctaggtaggcctcgagttatggcagttggttaaaaggaaacaaaaagaccgttttcacacaaaacggtctttttcgatttctttttacagtcacagccacttttgcaaaaaccggacagcttcatgccttataactgctgtttcggtcgacgaaacatcgttagatttcctcctaaattgacaaactaaatatctgataatttaacatattctcaaaagagtgtcaacgtgtattgacgcagtaaaggataaaagtaaagcctaataaatcaatgatctgacagcttgcaggtaatatatttaatttgaagcaattctctatacagccaaccagttatcgtttataatgtaattaaatttcatatgatcaatcttcggggcagggtgaaattccctaccggcggtgatgagccaatggctctaagcccgcgagctgtctttacagcaggattcggtgagattccggagccgacagtacagtctggatgggagaagatggaggttcataagcgttttgaaattgaatttttcaaacgtttctttgcctagcctaattttcgaaaccccgcttttatatatgaagcggtttttttattggctggaaaagaacctttccgttttcgagtaagatgtgatcgaaaaggagagaatgaagtgaaagtaaaaaaattagttgtggtcagcatgcaagcttcgcgaagcggccgccgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa